(19) United States
(12) Patent Application Publication
(10) Pub. No.: US 2022/0122692 A1
(43) Pub. Date: Apr. 21, 2022

(54) MACHINE LEARNING GUIDED POLYPEPTIDE ANALYSIS

(71) Applicant: Flagship Pioneering Innovations VI, LLC, Cambridge, MA (US)

(72) Inventors: Jacob D. Feala, Franklin, MA (US); Andrew Lane Beam, Jamaica Plain, MA (US); Molly Krisann Gibson, Medford, MA (US)

(21) Appl. No.: 17/428,356

(22) PCT Filed: Feb. 10, 2020
(86) PCT No.: PCT/US2020/017517

§ 371 Date: Aug. 4, 2021

Related U.S. Application Data
(60) Provisional application No. 62/804,034, filed on Feb. 11, 2019.
(60) Provisional application No. 62/804,036, filed on Feb. 11, 2019.

(51) Int. Cl. G16B 20/00; G16B 40/20; G16B 40/30; G06N 3/04; G06N 3/08

(52) U.S. Cl. CPC G16B 20/00; G16B 40/20; G06N 3/08; G06N 3/0454; G16B 40/30

(57) ABSTRACT

Systems, apparatuses, software, and methods for identifying associations between amino acid sequences and protein functions or properties. The application of machine learning is used to generate models that identify such associations based on input data such as amino acid sequence information. Various techniques including transfer learning can be utilized to enhance the accuracy of the associations.

### RELATED APPLICATIONS

This application claims the benefit of U.S. Provisional Application No. 62/804,034, filed on Feb. 11, 2019 and U.S. Provisional Application No. 62/804,036 filed Feb. 11, 2019. The entire teachings of the above applications are incorporated herein by reference.

### BACKGROUND

Proteins are macromolecules that are essential to living organisms and carry out or are associated with many functions within organisms, including, for example, catalyzing metabolic reactions, facilitating DNA replication, responding to stimuli, providing structure to cells and tissue, and transporting molecules. Proteins are made of one or more chains of amino acids and typically form three-dimensional conformations.

### SUMMARY

Described herein are systems, apparatuses, software, and methods for evaluating protein or polypeptide information and, in some embodiments, generating predictions of properties or functions. Protein properties and protein functions are a measurable value describing a phenotype. In practice protein function can refer to a primary therapeutic function and protein property can refer to other desired drug-like properties. In some embodiments of the systems, apparatuses, software, and methods described herein, a previously unknown relationship between an amino acid sequence and a protein function is identified.

Traditionally, protein function prediction based on an amino acid sequence is highly challenging due at least in part to the structural complexity that can arise from what is seemingly a simple primary amino acid sequence. The traditional approach is to apply statistical comparisons based on homology between proteins with known functions (or other similar approaches), which has failed to provide an accurate and reproducible method for predicting protein function based on an amino acid sequence.

In fact, traditional thinking with respect to protein prediction based on primary sequence (e.g., DNA, RNA, or amino acid sequence) is that a primary protein sequence cannot be directly associated with a known function, because so much of the proteins function is driven by its ultimate tertiary (or quaternary) structure.

In contrast to traditional approaches and traditional thinking with regard to protein analysis, the innovative systems, apparatuses, software, and methods described herein analyze an amino acid sequence using innovative machine learning techniques and/or advanced analytics to accurately and reproducibly identify previously unknown relationships between an amino acid sequence and a protein function. That is, the innovations described herein are unexpected and produce unexpected results in view of traditional thinking with respect to protein analysis and protein structure.

Described herein is a method of modeling a desired protein property comprising: (a) providing a first pretrained system comprising a neural net embedder and, optionally, a neural net predictor, the neural net predictor of the pretrained system being different from the desired protein property; (b) transferring at least a part of the neural net embedder of the pretrained system to a second system comprising a neural net embedder and a neural net predictor, the neural net predictor of the second system providing the desired protein property; and (c) analyzing, by the second system, the primary amino acid sequence of a protein analyte, thereby generating a prediction of the desired protein property for the protein analyte.

A person having ordinary skill in the art can recognize that in some embodiments, the primary amino acid sequence can be either a whole and partial amino acid sequence for a given protein analyte. In embodiments, the amino acid sequence can be continuous and discontinuous sequences. In embodiments, the amino acid sequence has at least 95% identity to a primary sequence of the protein analyte.

In some embodiments, the architecture of the neural net embedder of the first and second systems is a convolutional architecture independently selected from VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the first system comprises a generative adversarial network (GAN), recurrent neural network, or a variational autoencoder (VAE). In some embodiments, the first system comprises a generative adversarial network (GAN) selected from a conditional GAN, DCGAN, CGAN, SGAN or progressive GAN, SAGAN, LSGAN, WGAN, EBGAN, BEGAN, or infoGAN. In some embodiments, the first system comprises a recurrent neural network selected from a Bi-LSTM/LSTM, a Bi-GRU/GRU, or a transformer network. In some embodiments, the first system comprises a variational autoencoder (VAE). In some embodiments, the embedder is trained on a set of at least 50, 100, 150, 200, 250, 300, 350, 400, 450, 500, 600, 700, 800, 900, or 1000 or more amino acid sequences protein amino acid sequences. In some embodiments, the amino acid sequences include annotations across functional representations including at least one of GP, Pfam, keyword, Kegg Ontology, Interpro, SUPFAM, or OrthoDB. In some embodiments, the protein amino acid sequences have at least about 10, 20, 30, 40, 50, 75, 100, 120, 140, 150, 160, or 170 thousand possible annotations. In some embodiments, the second model has an improved performance metric relative to a model trained without using the transferred embedder of the first model. In some embodiments, the first or second systems are optimized by Adam, RMS prop, stochastic gradient descent (SGD) with momentum, SGD with momentum and Nestrov accelerated gradient, SGD without momentum, Adagrad, Adadelta, or NAdam. The first and the second model can be optimized using any of the follow activation functions: softmax, elu, SeLU, softplus, softsign, ReLU, tanh, sigmoid, hard_sigmoid, exponential, PReLU, and LeaskyReLU, or linear. In some embodiments, the neural net embedder comprises at least 10, 50, 100, 250, 500, 750, or 1000, or more layers, and the predictor comprises at least 1, 2, 3, 4, 5, 6, 7, 8, 9, 10, 11, 12, 13, 14, 15, 16, 17, 18, 19, or 20, or more layers. In some embodiments, at least one of the first or second system utilizes a regularization selected from: early stopping, L1-L2 regularization, skip connections, or a combination thereof, wherein the regularization is performed on 1, 2, 3, 4, 5, or more layers. In some embodiments, the regularization is performed using batch normalization. In some embodiments, the regularization is performed using group normalization. In some embodiments, a second model of the second system comprises a first model of the first system in which the last layer is removed. In some embodiments, 2, 3, 4, 5, or more layers of the first model are removed in a transfer to the second model. In some embodiments, the transferred layers are frozen during the training of the second model. In some embodiments, the transferred layers are unfrozen during the training of the second model. In some embodiments, the second model has 1, 2, 3, 4, 5, 6, 7, 8, 9, 10, or more layers added to the transferred layers of the first model. In some embodiments, the neural net predictor of the second system predicts one or more of protein binding activity, nucleic acid binding activity, protein solubility, and protein stability. In some embodiments, the neural net predictor of the second system predicts protein fluorescence. In some embodiments, the neural net predictor of the second system predicts enzymatic.

Described herein is a computer implemented method for identifying a previously unknown association between an amino acid sequence and a protein function comprising: (a) generating, with a first machine learning software module, a first model of a plurality of associations between a plurality of protein properties and a plurality of amino acid sequences; (b) transferring the first model or a portion thereof to a second machine learning software module; (c) generating, by the second machine learning software module, a second model comprising the first model or a portion thereof; and (d) identifying, based on the second model, the previously unknown association between the amino acid sequence and the protein function. In some embodiments, the amino acid sequence comprises a primary protein structure. In some embodiments, the amino acid sequence causes a protein configuration that results in the protein function. In some embodiments, the protein function comprises fluorescence. In some embodiments, the protein function comprises an enzymatic activity. In some embodiments, the protein function comprises nuclease activity. Example nuclease activities include restriction, endonuclease activity, and sequence guided endonuclease activity, such as Cas9 endonuclease activity. In some embodiments, the protein function comprises a degree of protein stability. In some embodiments, the plurality of protein properties and the plurality of amino acid sequences are from UniProt. In some embodiments, the plurality of protein properties comprise one or more of the labels GP, Pfam, keyword, Kegg Ontology, Interpro, SUPFAM, and OrthoDB. In some embodiments, the plurality of amino acid sequences include a primary protein structure, a secondary protein structure, and a tertiary protein structure for a plurality of proteins. In some embodiments, the amino acid sequences include sequences that can form a primary, secondary, and/or tertiary structure in a folded protein.

In some embodiments, the first model is trained on input data comprising one or more of a multidimensional tensor, a representation of 3-dimensional atomic positions, an adjacency matrix of pairwise interactions, and a character embedding. In some embodiments, the method comprises inputting to the second machine learning module, at least one of data related to a mutation of a primary amino acid sequence, a contact map of an amino acid interaction, a tertiary protein structure, and a predicted isoform from alternatively spliced transcripts. In some embodiments, the first model and the second model are trained using supervised learning. In some embodiments, the first model is trained using supervised learning, and the second model is trained using unsupervised learning. In some embodiments, the first model and the second model comprise a neural network comprising a convolutional neural network, a generative adversarial network, recurrent neural network, or a variational autoencoder. In some embodiments, the first model and the second model each comprise a different neural network architecture. In some embodiments, the convolutional network comprises one of VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the first model comprises an embedder and the second model comprises a predictor. In some embodiments, a first model architecture comprises a plurality of layers, and a second model architecture comprises at least two layers of the plurality of layers. In some embodiments, the first machine learning software module trains the first model on a first training data set comprising at least 10,000 protein properties and the second machine learning software module trains the second model using a second training data set.

Described herein is a computer system for identifying a previously unknown association between an amino acid sequence and a protein function comprising: (a) a processor; (b) a non-transitory computer readable medium encoded with software configured to cause the processor to: (i) generate, with a first machine learning software model, a first model of a plurality of associations between a plurality of protein properties and a plurality of amino acid sequences; (ii) transfer the first model or a portion thereof to a second machine learning software module; (iii) generate, by the second machine learning software module, a second model comprising the first model or a portion thereof; (iv) identify, based on the second model, the previously unknown association between the amino acid sequence and the protein function. In some embodiments, the amino acid sequence comprises a primary protein structure. In some embodiments, the amino acid sequence causes a protein configuration that results in the protein function. In some embodiments, the protein function comprises fluorescence. In some embodiments, the protein function comprises an enzymatic activity. In some embodiments, the protein function comprises nuclease activity. In some embodiments, the protein function comprises a degree of protein stability. In some embodiments, the plurality of protein properties and the plurality of protein markers are from UniProt. In some embodiments, the plurality of protein properties comprise one or more of the labels GP, Pfam, keyword, Kegg Ontology, Interpro, SUPFAM, and OrthoDB. In some embodiments, the plurality of amino acid sequences include a primary protein structure, a secondary protein structure, and a tertiary protein structure for a plurality of proteins. In some embodiments, the first model is trained on input data comprising one or more of a multidimensional tensor, a representation of 3-dimensional atomic positions, an adjacency matrix of pairwise interactions, and a character embedding. In some embodiments, the software is configured to cause the processor to input to the second machine learning module, at least one of data related to a mutation of a primary amino acid sequence, a contact map of an amino acid interaction, a tertiary protein structure, and a predicted isoform from alternatively spliced transcripts. In some embodiments, the first model and the second model are trained using supervised learning. In some embodiments, the first model is trained using supervised learning and the second model is trained using unsupervised learning. In some embodiments, the first model and the second model comprise a neural network comprising a convolutional neural network, a generative adversarial network, recurrent neural network, or a variational autoencoder. In some embodiments, the first model and the second model each comprise a different neural network architecture. In some embodiments, the convolutional network comprises one of VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, the first model comprises an embedder and the second model comprises a predictor. In some embodiments, a first model architecture comprises a plurality of layers and a second model architecture comprises at least two layers of the plurality of layers. In some embodiments, the first machine learning software module trains the first model on a first training data set comprising at least 10,000 protein properties and the second machine learning software module trains the second model using a second training data set.

In some embodiments, a method modeling a desired protein property includes training a first system with a first set of data. The first system includes a first neural net transformer encoder and a first decoder. The first decoder of the pretrained system is configured to generate an output different from the desired protein property. The method further includes transferring at least a part of the first transformer encoder of the pretrained system to a second system, the second system comprising a second transformer encoder and a second decoder. The method further includes training the second system with a second set of data. The second set of data includes a set of proteins representing a smaller number of classes of proteins than the first set, wherein the classes of proteins include one or more of: (a) classes of proteins within the first set of data, and (b) classes of proteins excluded from the first set of data. The method further includes analyzing, by the second system, a primary amino acid sequence of a protein analyte, thereby generating a prediction of the desired protein property for the protein analyte. In some embodiments, the second set of data can include either some overlapping data with the first set of data, or exclusively overlapping data with the first set of data. Alternatively, the second set of data has no overlapping data with the first set of data in some embodiments.

In some embodiments, the primary amino acid sequence of a protein analyte can be one or more Asparaginase sequences and corresponding activity labels. In some embodiments, the first set of data comprises a set of proteins including a plurality of classes of proteins. Example classes of proteins include structural proteins, contractile proteins, storage proteins, defensive proteins (e.g., antibodies), transport proteins, signal proteins, and enzymes proteins. Generally, the classes of proteins include proteins having amino acid sequences sharing one or more functional and/or structural similarities, and include the classes of proteins described below. A person having ordinary skill in the art can further understand that the classes can include groupings based on biophysical properties, such as solubility, structural features, secondary or tertiary motifs, thermostability, and other features known in the art. The second set of data can be one class of proteins, such as enzymes. In some embodiments, a system can be adapted for performing the above method.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an overview of the input block of a base deep learning model;

FIG. 2 shows an example of an identity block of a deep learning model;

FIG. 3 shows an example of a convolutional block of a deep learning model;

FIG. 4 shows an example of an output layer for a deep learning model;

FIG. 5 shows the expected vs. predicted stability of mini-proteins using a first model as described in Example 1 as the starting point and a second model as described in Example 2;

FIG. 6 shows the Pearson correlation of predicted vs measured data for different machine learning models as a function of the number of labeled protein sequences used in model training; the pretrained represents the method of the first model being used as a starting point for the second model as trained on specific protein function of fluorescence;

FIG. 7 shows the positive predictive power of different machine learning models as a function of the number of labeled protein sequences used in model training. The Pretrained (full model) represents the method of the first model being used as a starting point for the second model as trained on specific protein function of fluorescence;

FIG. 8 shows an embodiment of a system configured to perform the methods or functions of the present disclosure; and

FIG. 9 shows an embodiment of a process by which a first model is trained on annotated UniProt sequences and used to generate a second model through transfer learning.

FIG. 10A is block diagram illustrating an example embodiment of the present disclosure.

FIG. 10B is a block diagram illustrating an example embodiment of the method of the present disclosure.

FIG. 11 illustrates an example embodiment of splitting by antibody position.

FIG. 12 illustrates example results of linear, naïve, and pretrained transformer results using a random split and a split by position.

FIG. 13 is a graph illustrating reconstruction error for asparaginase sequences.

### DETAILED DESCRIPTION

A description of example embodiments follows.

Described herein are systems, apparatuses, software, and methods for evaluating protein or polypeptide information and, in some embodiments, generating predictions of properties or functions. Machine learning method allow for the generation of models that receive input data, such as a primary amino acid sequence, and predicting one or more functions or features of the resulting polypeptide or protein defined at least in part by the amino acid sequence. The input data can include additional information such as contact maps of amino acid interactions, tertiary protein structure, or other relevant information relating to the structure of the polypeptide. Transfer learning is used in some instances to improve the predictive ability of the model when there is insufficient labeled training data.

### Prediction of Polypeptide Properties or Functions

Described herein are devices, software, systems, and methods for evaluating input data comprising protein or polypeptide information such as amino acid sequences (or nucleic acid sequences that code for the amino acid sequences) in order to predict one or more specific functions or properties based on the input data. The extrapolation of specific function(s) or properties for amino acid sequences (e.g. proteins) would be beneficial for many molecular biology applications. Accordingly, the devices, software, systems, and methods described herein leverage the capabilities of artificial intelligence or machine learning techniques for polypeptide or protein analysis to make predictions about structure and/or function. Machine learning techniques enable the generation of models with increased predictive ability compared to standard non-ML approaches. In some cases, transfer learning is leveraged to enhance predictive accuracy when insufficient data is available to train the model for the desired output. Alternatively, in some cases, transfer learning is not utilized when there is sufficient data to train the model to achieve comparable statistical parameters as a model that incorporates transfer learning.

In some embodiments, input data comprises the primary amino acid sequence for a protein or polypeptide. In some cases, the models are trained using labeled data sets comprising the primary amino acid sequence. For example, the data set can include amino acid sequences of fluorescent proteins that are labeled based on the degree of fluorescence intensity. Accordingly, a model can be trained on this data set using a machine learning method to generate a prediction of fluorescence intensity for amino acid sequence inputs. In some embodiments, the input data comprises information in addition to the primary amino acid sequence such as, for example, surface charge, hydrophobic surface area, measured or predicted solubility, or other relevant information. In some embodiments, the input data comprises multi-dimensional input data including multiple types or categories of data.

In some embodiments, the devices, software, systems, and methods described herein utilize data augmentation to enhance performance of the predictive model(s). Data augmentation entails training using similar but different examples or variations of the training data set. As an example, in image classification, the image data can be augmented by slightly altering the orientation of the image (e.g., slight rotations). In some embodiments, the data inputs (e.g., primary amino acid sequence) are augmented by random mutation and/or biologically informed mutation to the primary amino acid sequence, multiple sequence alignments, contact maps of amino acid interactions, and/or tertiary protein structure. Additional augmentation strategies include the use of known and predicted isoforms from alternatively spliced transcripts. For example, input data can be augmented by including isoforms of alternatively spliced transcripts that correspond to the same function or property. Accordingly, data on isoforms or mutations can allow the identification of those portions or features of the primary sequence that do not significantly impact the predicted function or property. This allows a model to account for information such as, for example, amino acid mutations that enhance, decrease, or do not affect a predicted protein property such as stability. For example, data inputs can comprise sequences with random substituted amino acids at positions that are known not to affect function. This allows the models that are trained on this data to learn that the predicted function is invariant with respect to those particular mutations.

In some embodiments, data augmentation involves a “mixup” learning principle that entails training the network on convex combinations of example pairs and corresponding labels, as described in Zhang et al., Mixup: Beyond Empirical Risk Minimization, Arxiv 2018. This approach regularizes the network such that simple linear behavior between training samples is favored. Mixup provides a data-agnostic data augmentation process. In some embodiments, mixup data augmentation comprises generating virtual training examples or data according to the following formulas:

\tilde{x}+λxᵢ+(1−λ)xⱼ

\tilde{y}+λyᵢ+(1−λ)yⱼ

The parameters χᵢ and χⱼ are raw input vectors, and γᵢ and γⱼ are one-hot encodings. (χⱼ, γᵢ) and (χⱼ, γⱼ) are two examples or data inputs randomly selected from the training data set.

The devices, software, systems, and methods described herein can be used to generate a variety of predictions. The predictions can involve protein functions and/or properties (e.g., enzymatic activity, stability, etc.). Protein stability can be predicted according to various metrics such as, for example, thermostability, oxidative stability, or serum stability. Protein stability as defined by Rocklin can be considered one metric (e.g., susceptibility to protease cleavage) but another metric can be free energy of the folded (tertiary) structure. In some embodiments, a prediction comprises one or more structural features such as, for example, secondary structure, tertiary protein structure, quaternary structure, or any combination thereof. Secondary structure can include a designation of whether an amino acid or a sequence of amino acids in a polypeptide is predicted to have an alpha helical structure, a beta sheet structure, or a disordered or loop structure. Tertiary structure can include the location or positioning of amino acids or portions of the polypeptide in three-dimensional space. Quaternary structure can include the location or positioning of multiple polypeptides forming a single protein. In some embodiments, a prediction comprises one or more functions. Polypeptide or protein functions can belong to various categories including metabolic reactions, DNA replication, providing structure, transportation, antigen recognition, intracellular or extracellular signaling, and other functional categories. In some embodiments, a prediction comprises an enzymatic function such as, for example, catalytic efficiency (e.g., specificity constant k_{cat}/KM) or catalytic specificity.

In some embodiments, a prediction comprises an enzymatic function for a protein or polypeptide. In some embodiments, a protein function is an enzymatic function. Enzymes can perform various enzymatic reactions and can be categorized as transferases (e.g., transfers functional groups from one molecule to another), oxioreductases (e.g., catalyzes oxidation-reduction reactions), hydrolases (e.g., cleaves chemical bonds via hydrolysis), lyases (e.g., generate a double bond), ligases (e.g., joining two molecules via a covalent bond), and isomerases (e.g., catalyzes structural changes within a molecule from one isomer to another). In some embodiments, hydrolases include proteases such as serine proteases, threonine proteases, cysteine proteases, metalloproteases, asparagine peptide lyases, glutamic proteases, and aspartic proteases. Serine proteases have various physiological roles such as in blood coagulation, wound healing, digestion, immune responses and tumor invasion and metastasis. Examples of serine proteases include chymotrypsin, trypsin, elastase, Factor 10, Factor 11, Thrombin, Plasmin, C1r, C1s, and C3 convertases. Threonine proteases include a family of proteases that have a threonine within the active catalytic site. Examples of threonine proteases include subunits of the proteasome. The proteasome is a barrel-shaped protein complex made up of alpha and beta subunits. The catalytically active beta subunit can include a conserved N-terminal threonine at each active site for catalysis. Cysteine proteases have a catalytic mechanism that utilizes a cysteine sulfhydryl group. Examples of cysteine proteases include papain, cathepsin, caspases, and calpains. Aspartic proteases have two aspartate residues that participate in acid/base catalysis at the active site. Examples of aspartatic proteases include the digestive enzyme pepsin, some lysosomal proteases, and renin. Metalloproteases include the digestive enzymes carboxypeptidases, matrix metalloproteases (MMPs) which play roles in extracellular matrix remodeling and cell signaling, ADAMs (a disintegrin and metalloprotease domain), and lysosomal proteases. Other non-limiting examples of enzymes include proteases, nucleases, DNA ligases, polymerases, cellulases, liginases, amylases, lipases, pectinases, xylanases, lignin peroxidases, decarboxylases, mannanases, dehydrogenases, and other polypeptide-based enzymes.

In some embodiments, enzymatic reactions include post-translational modifications of target molecules. Examples of post-translational modifications include acetylation, amidation, formylation, glycosylation, hydroxylation, methylation, myristoylation, phosphorylation, deamidation, prenylation (e.g., farnesylation, geranylation, etc.), ubiquitylation, ribosylation and sulphation. Phosphorylation can occur on an amino acid such as tyrosine, serine, threonine, or histidine.

In some embodiments, the protein function is luminescence which is light emission without requiring the application of heat. In some embodiments, the protein function is chemiluminescence such as bioluminescence. For example, a chemiluminescent enzyme such as luciferin can act on a substrate (luciferin) to catalyze the oxidation of the substrate, thereby releasing light. In some embodiments, the protein function is fluorescence in which the fluorescent protein or peptide absorbs light of certain wavelength(s) and emits light at different wavelength(s). Examples of fluorescent proteins include green fluorescent protein (GFP) or derivatives of GFP such as EBFP, EBFP2, Azurite, mKalama1, ECFP, Cerulean, CyPet, YFP, Citrine, Venus, or YPet. Some proteins such as GFP are naturally fluorescent. Examples of fluorescent proteins include EGFP, blue fluorescent protein (EBFP, EBFP2, Azurite, mKalama1), cyan fluorescent protein (ECFP, Cerulean, CyPet), yellow fluorescent protein (YFP, Citrine, Venus, YPet), redox sensitive GFP (roGFP), and monomeric GFP.

In some embodiments, the protein function comprises an enzymatic function, binding (e.g., DNA/RNA binding, protein binding, etc.), immune function (e.g., antibody), contraction (e.g., actin, myosin), and other functions. In some embodiments, the output comprises a value associated with the protein function such as, for example, kinetics of enzymatic function or binding. Such outputs can include metrics for affinity, specificity, and reaction rate.

In some embodiments, the machine learning method(s) described herein comprise supervised machine learning. Supervised machine learning includes classification and regression. In some embodiments, the machine learning method(s) comprise unsupervised machine learning. Unsupervised machine learning includes clustering, autoencoding, variational autoencoding, protein language model (e.g., wherein the model predicts the next amino acid in a sequence when given access to the previous amino acids), and association rules mining.

In some embodiments, a prediction comprises a classification such as a binary, multi-label, or multi-class classification. The prediction can be of a protein property, in some embodiments. Classifications are generally used to predict a discrete class or label based on input parameters.

A binary classification predicts which of two groups a polypeptide or protein belongs in based on the input. In some embodiments, a binary classification includes a positive or negative prediction for a property or function for a protein or polypeptide sequence. In some embodiments, a binary classification includes any quantitative readout subject to a threshold such as, for example, binding to a DNA sequence above some level of affinity, catalyzing a reaction above some threshold of kinetic parameter, or exhibiting thermostability above a certain melting temperature. Examples of a binary classification include positive/negative predictions that a polypeptide sequence exhibits autofluorescence, is a serine protease, or is a GPI-anchored transmembrane protein.

In some embodiments, the classification (of the prediction) is a multi-class classification or multi-label classification. For example, a multi-class classification can categorize input polypeptides into one of more than two mutually exclusive groups or categories, whereas multi-label classification classifies input into multiple labels or groups. For example, multi-label classification may label a polypeptide as being both a intracellular protein (vs extracellular) and a protease. By comparison, multi-class classification may include classifying an amino acid as belonging to one of an alpha helix, a beta sheet, or a disordered/loop peptide sequence. Therefore, protein properties can include exhibiting autofluorescence, being a serine protease, being a GPI-anchored transmembrane protein, being a intracellular protein (vs extracellular) and/or a protease, and belonging to an alpha helix, a beta sheet, or a disordered/loop peptide sequence.

In some embodiments, a prediction comprises a regression that provides a continuous variable or value such as, for example, the intensity of auto-fluorescence or the stability of a protein. In some embodiments, the prediction comprises a continuous variable or value for any of the properties or functions described herein. As an example, the continuous variable or value can be indicative of the targeting specificity of a matrix metalloprotease for a particular substrate extracellular matrix component. Additional examples include various quantitative readouts such as target molecule binding affinity (e.g., DNA binding), reaction rate of an enzyme, or thermostability.

### Machine Learning Method

Described herein are devices, software, systems, and methods that apply one or more methods for analyzing input data to generate predictions relating to one or more protein or polypeptide properties or functions. In some embodiments, the methods utilize statistical modeling to generate predictions or estimates about protein or polypeptide function(s) or properties. In some embodiments, machine learning methods are used for training prediction models and/or making predictions. In some embodiments, the method predicts a likelihood or probability of one or more properties or functions. In some embodiments, a method utilizes a predictive model such as a neural network, a decision tree, a support vector machine, or other applicable model. Using the training data, a method forms a classifier for generating a classification or prediction according to relevant features. The features selected for classification can be classified using a variety of methods. In some embodiments, the trained method comprises a machine learning method.

In some embodiments, the machine learning method uses a support vector machine (SVM), a Naïve Bayes classification, a random forest, or an artificial neural network. Machine learning techniques include bagging procedures, boosting procedures, random forest methods, and combinations thereof. In some embodiments, the predictive model is a deep neural network. In some embodiments, the predictive model is a deep convolutional neural network.

In some embodiments, a machine learning method uses a supervised learning approach. In supervised learning, the method generates a function from labeled training data. Each training example is a pair including an input object and a desired output value. In some embodiments, an optimal scenario allows for the method to correctly determine the class labels for unseen instances. In some embodiments, a supervised learning method requires the user to determine one or more control parameters. These parameters are optionally adjusted by optimizing performance on a subset, called a validation set, of the training set. After parameter adjustment and learning, the performance of the resulting function is optionally measured on a test set that is separate from the training set. Regression methods are commonly used in supervised learning. Accordingly, supervised learning allows for a model or classifier to be generated or trained with training data in which the expected output is known in advance such as in calculating a protein function when the primary amino acid sequence is known.

In some embodiments, a machine learning method uses an unsupervised learning approach. In unsupervised learning, the method generates a function to describe hidden structures from unlabeled data (e.g., a classification or categorization is not included in the observations). Since the examples given to the learner are unlabeled, there is no evaluation of the accuracy of the structure that is output by the relevant method. Approaches to unsupervised learning include: clustering, anomaly detection, and approaches based on neural networks including autoencoders and variational autoencoders.

In some embodiments, the machine learning method utilizes multi-class learning. Multi-task learning (MTL) is an area of machine learning in which more than one learning task is solved simultaneously in a manner that takes advantage of commonalities and differences across the multiple tasks. Advantages of this approach can include improved learning efficiency and prediction accuracy for the specific predictive models in comparison to training those models separately. Regularization to prevent overfitting can be provided by requiring an method to perform well on a related task. This approach can be better than regularization that applies an equal penalty to all complexity. Multi-class learning can be especially useful when applied to tasks or predictions that share significant commonalities and/or are under-sampled. In some embodiments, multi-class learning is effective for tasks that do not share significant commonalities (e.g., unrelated tasks or classifications). In some embodiments, multi-class learning is used in combination with transfer learning.

In some embodiments, a machine learning method learns in batches based on the training dataset and other inputs for that batch. In other embodiments, the machine learning method performs additional learning where the weights and error calculations are updated, for example, using new or updated training data. In some embodiments, the machine learning method updates the prediction model based on new or updated data. For example, a machine learning method can be applied to new or updated data to be re-trained or optimized to generate a new prediction model. In some embodiments, a machine learning method or model is re-trained periodically as additional data becomes available.

In some embodiments, the classifier or trained method of the present disclosure comprises one feature space. In some cases, the classifier comprises two or more feature spaces. In some embodiments, the two or more feature spaces are distinct from one another. In some embodiments, the accuracy of the classification or prediction is improved by combining two or more feature spaces in a classifier instead of using a single feature space. The attributes generally make up the input features of the feature space and are labeled to indicate the classification of each case for the given set of input features corresponding to that case.

The accuracy of the classification may be improved by combining two or more feature spaces in a predictive model or classifier instead of using a single feature space. In some embodiments, the predictive model comprises at least two, three, four, five, six, seven, eight, nine, or ten or more feature spaces. The polypeptide sequence information and optionally additional data generally make up the input features of the feature space and are labeled to indicate the classification of each case for the given set of input features corresponding to that case. In many cases, the classification is the outcome of the case. The training data is fed into the machine learning method which processes the input features and associated outcomes to generate a trained model or predictor. In some cases, the machine learning method is provided with training data that includes the classification, thus enabling the method to “learn” by comparing its output with the actual output to modify and improve the model. This is often referred to as supervised learning. Alternatively, in some instances, the machine learning method is provided with unlabeled or unclassified data, which leaves the method to identify hidden structure amongst the cases (e.g., clustering). This is referred to as unsupervised learning.

In some embodiments, one or more sets of training data are used to train a model using a machine learning method. In some embodiments, the methods described herein comprise training a model using a training data set. In some embodiments, the model is trained using a training data set comprising a plurality of amino acid sequences. In some embodiments, the training data set comprises at least 1, 2, 3, 4, 5, 6, 7, 8, 9, 10, 15, 20, 25, 30, 35, 40, 45, 50, 55, 56, 57, 58 million protein amino acid sequences. In some embodiments, the training data set comprises at least 10, 20, 30, 40, 50, 60, 70, 80, 90, 100, 150, 200, 250, 300, 350, 400, 450, 500, 600, 700, 800, 900, or 1000 or more amino acid sequences. In some embodiments, the training data set comprises at least 50, 100, 200, 300, 400, 500, 600, 700, 800, 900, 1000, 2000, 3000, 4000, 5000, 6000, 7000, 8000, 9000, or 10000 or more annotations. Although example embodiments of the present disclosure include machine learning methods that use deep neural networks, various types of methods are contemplated. In some embodiments, the method utilizes a predictive model such as a neural network, a decision tree, a support vector machine, or other applicable model. In some embodiments, the machine learning method is selected from the group including a supervised, semi-supervised and unsupervised learning, such as, for example, a support vector machine (SVM), a Naïve Bayes classification, a random forest, an artificial neural network, a decision tree, a K-means, learning vector quantization (LVQ), self-organizing map (SOM), graphical model, regression method (e.g., linear, logistic, multivariate, association rule learning, deep learning, dimensionality reduction and ensemble selection methods. In some embodiments, the machine learning method is selected from the group including: a support vector machine (SVM), a Naïve Bayes classification, a random forest, and an artificial neural network. Machine learning techniques include bagging procedures, boosting procedures, random forest methods, and combinations thereof. Illustrative methods for analyzing the data include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

### Transfer Learning

Described herein are devices, software, systems, and methods for predicting one or more protein or polypeptide properties or functions based on information such as primary amino acid sequence. In some embodiments, transfer learning is used to enhance predictive accuracy. Transfer learning is a machine learning technique where a model developed for one task can be reused as the starting point for a model on a second task. Transfer learning can be used to boost predictive accuracy on a task where there is limited data by having the model learn a on a related task where data is abundant. Accordingly, described herein are methods for learning general, functional features of proteins from a large data set of sequenced proteins and using it as a starting point for a model to predict any specific protein function, property, or feature. The present disclosure recognizes the surprising discovery that the information encoded in all sequenced proteins by a first predictive model can be transferred to design specific protein functions of interest using a second predictive model. In some embodiments, the predictive models are neural networks such as, for example, deep convolutional neural networks.

The present disclosure can be implemented via one or more embodiments to achieve one or more of the following advantages. In some embodiments, a prediction module or predictor trained with transfer learning exhibits improvements from a resource consumption standpoint such as exhibiting a small memory footprint, low latency, or low computational cost. This advantage cannot be understated in complex analyses that can require tremendous computing power. In some cases, the use of transfer learning is necessary to train sufficiently accurate predictors within a reasonable period of time (e.g., days instead of weeks). In some embodiments, the predictor trained using transfer learning provides a high accuracy compared to a predictor not trained using transfer learning. In some embodiments, the use of a deep neural network and/or transfer learning in a system for predicting polypeptide structure, property, and/or function increases computational efficiency compared to other methods or models that do not use transfer learning.

Described herein are methods of modeling a desired protein function or property. In some embodiments, a first system is provided comprising a neural net embedder. In some embodiments, the neural net embedder comprises one or more embedding layers. In some embodiments, the input to the neural network comprises a protein sequence represented as a “one-hot” vector that encodes the sequence of amino acids as a matrix. For example, within the matrix, each row can be configured to contain exactly 1 non-zero entry which corresponds to the amino acid present at that residue. In some embodiments, the first system comprises a neural net predictor. In some embodiments, the predictor comprises one or more output layers for generating a prediction or output based on the input. In some embodiments, the first system is pretrained using a first training data set to provide a pretrained neural net embedder. With transfer learning, the pretrained first system or a portion thereof can be transferred to form part of a second system. The one or more layers of the neural net embedder can be frozen when used in the second system. In some embodiments, the second system comprises the neural net embedder or a portion thereof from the first system. In some embodiments, the second system comprises a neural net embedder and a neural net predictor. The neural net predictor can include one or more output layers for generating a final output or prediction. The second system can be trained using a second training data set that is labeled according to the protein function or property of interest. As used herein, an embedder and a predictor can refer to components of a predictive model such as neural net trained using machine learning.

In some embodiments, transfer learning is used to train a first model, at least part of which is used to form a portion of a second model. The input data to the first model can comprise a large data repository of known natural and synthetic proteins, regardless of function or other properties. The input data can include any combination of the following: primary amino acid sequence, secondary structure sequences, contact maps of amino acid interactions, primary amino acid sequence as a function of amino acid physicochemical properties, and/or tertiary protein structures. Although these specific examples are provided herein, any additional information relating to the protein or polypeptide is contemplated. In some embodiments, the input data is embedded. For example, the input data can be represented as a multidimensional tensor of binary 1-hot encodings of sequences, real-values (e.g., in the case of physicochemical properties or 3-dimensional atomic positions from tertiary structure), adjacency matrices of pairwise interactions, or using a direct embedding of the data (e.g., character embeddings of the primary amino acid sequence).

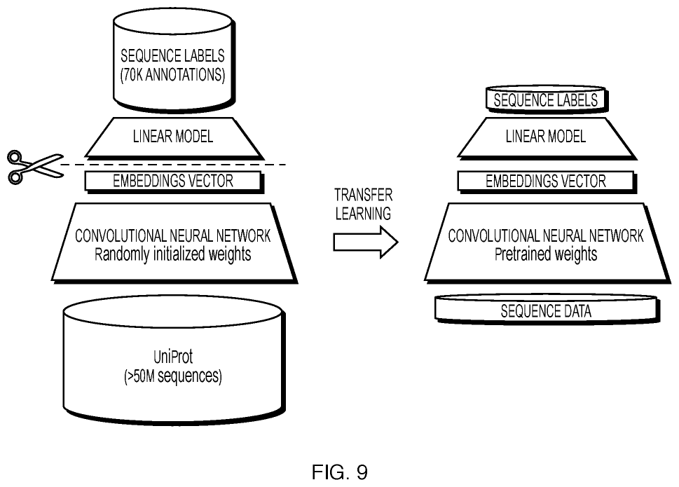

FIG. 9 is a block diagram illustrating an embodiment of the transfer learning process as applied to a neural network architecture. As shown, a first system (left) has a convolutional neural network architecture with an embedding vector and linear model that is trained using UniProt amino acid sequences and ˜70,000 annotations (e.g., sequence labels). During the transfer learning process, the embedding vector and convolutional neural network portion of the first system or model are transferred to form the core of a second system or model that also incorporates a new linear model configured to predict a protein property or function different from any prediction configured in the first model or system. This second system, having a linear model separate from the first system, is trained using a second training data set based on the desired sequence labels corresponding to the protein property or function. Once training is finished, the second system can be assessed against a validation data set and/or a test data set (e.g., data not used in training) and, once validated, can be used to analyze sequences for protein properties or functions. Protein properties can be used, for example, in therapeutic applications. Therapeutic applications can sometimes require a protein to have multiple drug-like properties, including stability, solubility, and expression (e.g., for manufacturing) in addition to its primary therapeutic function (e.g., catalysis for an enzyme, binding affinity for an antibody, stimulation of a signaling pathway for a hormone, etc.).

In some embodiments, the data inputs to the first model and/or the second model are augmented by additional data such as random mutation and/or biologically informed mutation to the primary amino acid sequence, contact maps of amino acid interactions, and/or tertiary protein structure. Additional augmentation strategies include the use of known and predicted isoforms from alternatively spliced transcripts. In some embodiments, different types of inputs (e.g., amino acid sequence, contact maps, etc.) are processed by different portions of one or more models. After the initial processing steps, the information from multiple data sources can be combined at a layer in the network. For example, a network can comprise a sequence encoder, a contact map encoder, and other encoders configured to receive and/or process various types of data inputs. In some embodiments, the data is turned into an embedding within one or more layers in the network.

The labels for the data inputs to the first model can be drawn from one or more public protein sequence annotations resources such as, for example: Gene Ontology (GO), Pfam domains, SUPFAM domains, Enzyme Commission (EC) numbers, taxonomy, extremophile designation, keywords, ortholog group assignments including OrthoDB and KEGG Ortholog. In addition, labels can be assigned based on known structural or fold classifications designated by databases such as SCOP, FSSP, or CATH, including all-α, all-β, α+β, α/β, membrane, intrinsically disordered, coiled coil, small, or designed proteins. For proteins for which the structure is known, quantitative global characteristics such as total surface charge, hydrophobic surface area, measured or predicted solubility, or other numeric quantities can be used as additional labels fit by a predictive model such as a multi-task model. Although these inputs are described in the context of transfer learning, the application of these inputs for non-transfer learning approaches is also contemplated. In some embodiments, the first model comprises an annotation layer that is stripped away to leave the core network composed of the encoder. The annotation layer can include multiple independent layers, each corresponding to a particular annotation such as, for example, primary amino acid sequence, GO, Pfam, Interpro, SUPFAM, KO, OrthoDB, and keywords. In some embodiments, the annotation layer comprises at least 1, 2, 3, 4, 5, 6, 7, 8, 9, 10, 15, 20, 25, 30, 40, 50, 60, 70, 80, 90, 100, 1000, 5000, 10000, 50000, 100000, or 150000 or more independent layers. In some embodiments, the annotation layer comprises 180000 independent layers. In some embodiments, a model is trained using at least 1, 2, 3, 4, 5, 6, 7, 8, 9, 10, 15, 20, 25, 30, 40, 50, 60, 70, 80, 90, 100, 1000, 5000, 10000, 50000, 100000, or 150000 or more annotations. In some embodiments, a model is trained using about 180000 annotations. In some embodiments, the model is trained with multiple annotations across a plurality of functional representations (e.g., one or more of GO, Pfam, keywords, Kegg Ontology, Interpro, SUPFAM, and OrthoDB). Amino acid sequence and annotation information can be obtained from various databases such as UniProt.

In some embodiments, the first model and the second model comprise a neural network architecture. The first model and the second model can be a supervised model using a convolutional architecture in the form of a 1D convolution (e.g. primary amino acid sequence), a 2D convolution (e.g. contact maps of amino acid interactions), or a 3D convolution (e.g. tertiary protein structures). The convolutional architecture can be one of the following described architectures: VGG16, VGG19, Deep ResNet, Inception/GoogLeNet (V1-V4), Inception/GoogLeNet ResNet, Xception, AlexNet, LeNet, MobileNet, DenseNet, NASNet, or MobileNet. In some embodiments, a single model approach (e.g., non-transfer learning) is contemplated that utilizes any of the architectures described herein.

The first model can also be an unsupervised model using either a generative adversarial network (GAN), recurrent neural network, or a variational autoencoder (VAE). If a GAN, the first model can be a conditional GAN, deep convolutional GAN, StackGAN, infoGAN, Wasserstein GAN, Discover Cross-Domain Relations with Generative Adversarial Networks (Disco GANS). In the case of a recurrent neural network, the first model can be a Bi-LSTM/LSTM, a Bi-GRU/GRU, or a transformer network. In some embodiments, a single model approach (e.g., non-transfer learning) is contemplated that utilizes any of the architectures described herein. In some embodiments, a GAN is DCGAN, CGAN, SGAN/progressive GAN, SAGAN, LSGAN, WGAN, EBGAN, BEGAN, or infoGAN. A recurrent neural network (RNN) is a variant of a tradition neural network built for sequential data. LSTM refers to long short term memory, which is a type of neuron in an RNN with a memory that allows it to model sequential or temporal dependencies in data. GRU refers to gated recurrent unit, which is a variant of the LSTM which attempts to address some the LSTMs shortcomings. Bi-LSTM/Bi-GRU refers to “bidirectional” variants of LSTM and GRU. Typically LSTMs and GRUs process sequential in the “forward” direction, but bi-directional versions learn in the “backward” direction as well. LSTM enables the preservation of information from data inputs that have already passed through it using the hidden state. Unidirectional LSTM only preserves information of the past because it has only seen inputs from the past. By contrast, bidirectional LSTM runs the data inputs in both directions from the past to the future and vice versa. Accordingly, the bidirectional LSTM that runs forwards and backwards preserves information from the future and the past.

For both the first model and the second model and supervised and unsupervised models, they can have alternative regularization methods, including early stopping, including drop outs at 1, 2, 3, 4, up to all layers, including L1-L2 regularization on 1, 2, 3, 4, up to all layers, including skip connections at 1, 2, 3, 4, up to all layers. For both the first model and the second model, regularization can be performed using batch normalization or group normalization. L1 regularization (also known as the LASSO) controls how long the L1 norm of the weight vector is allowed to be, whereas L2 controls how large the L2 norm can be. Skip connections can be obtained from the Resnet architecture.

The first and the second model can be optimized using any of the following optimization procedures: Adam, RMS prop, stochastic gradient descent (SGD) with momentum, SGD with momentum and Nestrov accelerated gradient, SGD without momentum, Adagrad, Adadelta, or NAdam. The first and the second model can be optimized using any of the follow activation functions: softmax, elu, SeLU, softplus, softsign, ReLU, tanh, sigmoid, hard_sigmoid, exponential, PReLU, and LeaskyReLU, or linear. In some embodiments, the methods described herein comprise “reweighting” the loss function that the optimizers listed above attempt to minimize, so that approximately equal weight is placed on both positive and negative examples. For example, one of the 180,000 outputs predicts the probability that a given protein is a membrane protein. Since a protein can only be a membrane protein or not a membrane protein, this is binary classification task, and the traditional loss function for a binary classification task is “binary cross-entropy”: loss(p,y)=−y*log(p)−(1−y)*log(1−p), where p is the probability of being a membrane protein according to the network and y is the “label” which is 1 if the protein is a membrane protein and 0 if it is not. A problem may arise if there are far more examples of y=0 because the network can likely learn the pathological rule of always predicting extremely low probabilities for this annotation because it is rarely penalized for always predicting y=0. To solve this problem, in some embodiments, the loss function is modified to the following: loss(p,y)=−w1*y*log(p)−w0*(1−y)*log(1−p), where w1 is the weight for the positive class and w0 is the weight for the negative class. This approach assumes w0=1 and w1=1/√((1−f0)/f1), where f0 is the frequency of negative examples and f1 is the frequency of positive examples. This weighting scheme “upweights” the positive examples which are rare, and “downweights” the negative examples which are more common.

The second model can use the first model as a starting point for training. The starting point can be the full first model frozen except the output layer, which is trained on the target protein function or protein property. The starting point can be the first model where the embedding layer, last 2 layers, last 3 layers, or all layers are unfrozen and the rest of the model is frozen during training on the target protein function or protein property. The starting point can be the first model where the embedding layer is removed and 1, 2, 3, or more layers are added and trained on the target protein function or protein property. In some embodiments, the number of frozen layers is 1 to 10. In some embodiments, the number of frozen layers is 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10. In some embodiments, the number of frozen layers is 1, 2, 3, 4, 5, 6, 7, 8, 9, or 10. In some embodiments, the number of frozen layers is at least 1, 2, 3, 4, 5, 6, 7, 8, or 9. In some embodiments, the number of frozen layers is at most 2, 3, 4, 5, 6, 7, 8, 9, or 10. In some embodiments, no layers are frozen during transfer learning. In some embodiments, the number of layers that are frozen in the first model is determined at least partly based on the number of samples available for training the second model. The present disclosure recognizes that freezing layer(s) or increasing the number of frozen layers can enhance the predictive performance of the second model. This effect can be accentuated in the case of low sample size for training the second model. In some embodiments, all the layers from the first model are frozen when the second model has no more than 200, 190, 180, 170, 160, 150, 140, 130, 120, 110, 100, 90, 80, 70, 60, 50, 40, or 30 samples in a training set. In some embodiments, at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or at least 100 layers in the first model are frozen for transfer to the second model when the number of samples for training the second model is no more than 200, 190, 180, 170, 160, 150, 140, 130, 120, 110, 100, 90, 80, 70, 60, 50, 40, or 30 samples in a training set.

The first and the second model can have 10-100 layers, 100-500 layers, 500-1000 layers, 1000-10000 layers, or up to 1000000 layers. In some embodiments, the first and/or second model comprises 10 layers to 1,000,000 layers. In some embodiments, the first and/or second model comprises 10 layers to 50 layers, 10 layers to 100 layers, 10 layers to 200 layers, 10 layers to 500 layers, 10 layers to 1,000 layers, 10 layers to 5,000 layers, 10 layers to 10,000 layers, 10 layers to 50,000 layers, 10 layers to 100,000 layers, 10 layers to 500,000 layers, 10 layers to 1,000,000 layers, 50 layers to 100 layers, 50 layers to 200 layers, 50 layers to 500 layers, 50 layers to 1,000 layers, 50 layers to 5,000 layers, 50 layers to 10,000 layers, 50 layers to 50,000 layers, 50 layers to 100,000 layers, 50 layers to 500,000 layers, 50 layers to 1,000,000 layers, 100 layers to 200 layers, 100 layers to 500 layers, 100 layers to 1,000 layers, 100 layers to 5,000 layers, 100 layers to 10,000 layers, 100 layers to 50,000 layers, 100 layers to 100,000 layers, 100 layers to 500,000 layers, 100 layers to 1,000,000 layers, 200 layers to 500 layers, 200 layers to 1,000 layers, 200 layers to 5,000 layers, 200 layers to 10,000 layers, 200 layers to 50,000 layers, 200 layers to 100,000 layers, 200 layers to 500,000 layers, 200 layers to 1,000,000 layers, 500 layers to 1,000 layers, 500 layers to 5,000 layers, 500 layers to 10,000 layers, 500 layers to 50,000 layers, 500 layers to 100,000 layers, 500 layers to 500,000 layers, 500 layers to 1,000,000 layers, 1,000 layers to 5,000 layers, 1,000 layers to 10,000 layers, 1,000 layers to 50,000 layers, 1,000 layers to 100,000 layers, 1,000 layers to 500,000 layers, 1,000 layers to 1,000,000 layers, 5,000 layers to 10,000 layers, 5,000 layers to 50,000 layers, 5,000 layers to 100,000 layers, 5,000 layers to 500,000 layers, 5,000 layers to 1,000,000 layers, 10,000 layers to 50,000 layers, 10,000 layers to 100,000 layers, 10,000 layers to 500,000 layers, 10,000 layers to 1,000,000 layers, 50,000 layers to 100,000 layers, 50,000 layers to 500,000 layers, 50,000 layers to 1,000,000 layers, 100,000 layers to 500,000 layers, 100,000 layers to 1,000,000 layers, or 500,000 layers to 1,000,000 layers. In some embodiments, the first and/or second model comprises 10 layers, 50 layers, 100 layers, 200 layers, 500 layers, 1,000 layers, 5,000 layers, 10,000 layers, 50,000 layers, 100,000 layers, 500,000 layers, or 1,000,000 layers. In some embodiments, the first and/or second model comprises at least 10 layers, 50 layers, 100 layers, 200 layers, 500 layers, 1,000 layers, 5,000 layers, 10,000 layers, 50,000 layers, 100,000 layers, or 500,000 layers. In some embodiments, the first and/or second model comprises at most 50 layers, 100 layers, 200 layers, 500 layers, 1,000 layers, 5,000 layers, 10,000 layers, 50,000 layers, 100,000 layers, 500,000 layers, or 1,000,000 layers.

In some embodiments, described herein is a first system comprises a neural net embedder and optionally a neural net predictor. In some embodiments, a second system comprises a neural net embedder and a neural net predictor. In some embodiments, the embedder comprises 10 layers to 200 layers. In some embodiments, the embedder comprises 10 layers to 20 layers, 10 layers to 30 layers, 10 layers to 40 layers, 10 layers to 50 layers, 10 layers to 60 layers, 10 layers to 70 layers, 10 layers to 80 layers, 10 layers to 90 layers, 10 layers to 100 layers, 10 layers to 200 layers, 20 layers to 30 layers, 20 layers to 40 layers, 20 layers to 50 layers, 20 layers to 60 layers, 20 layers to 70 layers, 20 layers to 80 layers, 20 layers to 90 layers, 20 layers to 100 layers, 20 layers to 200 layers, 30 layers to 40 layers, 30 layers to 50 layers, 30 layers to 60 layers, 30 layers to 70 layers, 30 layers to 80 layers, 30 layers to 90 layers, 30 layers to 100 layers, 30 layers to 200 layers, 40 layers to 50 layers, 40 layers to 60 layers, 40 layers to 70 layers, 40 layers to 80 layers, 40 layers to 90 layers, 40 layers to 100 layers, 40 layers to 200 layers, 50 layers to 60 layers, 50 layers to 70 layers, 50 layers to 80 layers, 50 layers to 90 layers, 50 layers to 100 layers, 50 layers to 200 layers, 60 layers to 70 layers, 60 layers to 80 layers, 60 layers to 90 layers, 60 layers to 100 layers, 60 layers to 200 layers, 70 layers to 80 layers, 70 layers to 90 layers, 70 layers to 100 layers, 70 layers to 200 layers, 80 layers to 90 layers, 80 layers to 100 layers, 80 layers to 200 layers, 90 layers to 100 layers, 90 layers to 200 layers, or 100 layers to 200 layers. In some embodiments, the embedder comprises 10 layers, 20 layers, 30 layers, 40 layers, 50 layers, 60 layers, 70 layers, 80 layers, 90 layers, 100 layers, or 200 layers. In some embodiments, the embedder comprises at least 10 layers, 20 layers, 30 layers, 40 layers, 50 layers, 60 layers, 70 layers, 80 layers, 90 layers, or 100 layers. In some embodiments, the embedder comprises at most 20 layers, 30 layers, 40 layers, 50 layers, 60 layers, 70 layers, 80 layers, 90 layers, 100 layers, or 200 layers.

In some embodiments, the neural net predictor comprises a plurality of layers. In some embodiments, the embedder comprises 1 layer to 20 layers. In some embodiments, the embedder comprises 1 layer to 2 layers, 1 layer to 3 layers, 1 layer to 4 layers, 1 layer to 5 layers, 1 layer to 6 layers, 1 layer to 7 layers, 1 layer to 8 layers, 1 layer to 9 layers, 1 layer to 10 layers, 1 layer to 15 layers, 1 layer to 20 layers, 2 layers to 3 layers, 2 layers to 4 layers, 2 layers to 5 layers, 2 layers to 6 layers, 2 layers to 7 layers, 2 layers to 8 layers, 2 layers to 9 layers, 2 layers to 10 layers, 2 layers to 15 layers, 2 layers to 20 layers, 3 layers to 4 layers, 3 layers to 5 layers, 3 layers to 6 layers, 3 layers to 7 layers, 3 layers to 8 layers, 3 layers to 9 layers, 3 layers to 10 layers, 3 layers to 15 layers, 3 layers to 20 layers, 4 layers to 5 layers, 4 layers to 6 layers, 4 layers to 7 layers, 4 layers to 8 layers, 4 layers to 9 layers, 4 layers to 10 layers, 4 layers to 15 layers, 4 layers to 20 layers, 5 layers to 6 layers, 5 layers to 7 layers, 5 layers to 8 layers, 5 layers to 9 layers, 5 layers to 10 layers, 5 layers to 15 layers, 5 layers to 20 layers, 6 layers to 7 layers, 6 layers to 8 layers, 6 layers to 9 layers, 6 layers to 10 layers, 6 layers to 15 layers, 6 layers to 20 layers, 7 layers to 8 layers, 7 layers to 9 layers, 7 layers to 10 layers, 7 layers to 15 layers, 7 layers to 20 layers, 8 layers to 9 layers, 8 layers to 10 layers, 8 layers to 15 layers, 8 layers to 20 layers, 9 layers to 10 layers, 9 layers to 15 layers, 9 layers to 20 layers, 10 layers to 15 layers, 10 layers to 20 layers, or 15 layers to 20 layers. In some embodiments, the embedder comprises 1 layer, 2 layers, 3 layers, 4 layers, 5 layers, 6 layers, 7 layers, 8 layers, 9 layers, 10 layers, 15 layers, or 20 layers. In some embodiments, the embedder comprises at least 1 layer, 2 layers, 3 layers, 4 layers, 5 layers, 6 layers, 7 layers, 8 layers, 9 layers, 10 layers, or 15 layers. In some embodiments, the embedder comprises at most 2 layers, 3 layers, 4 layers, 5 layers, 6 layers, 7 layers, 8 layers, 9 layers, 10 layers, 15 layers, or 20 layers.

In some embodiments, transfer learning is not used to generate the final trained model. For example, in cases when sufficient data is available, a model generated at least in part using transfer learning does not provide a significant improvement in predictions compared to a model that does not utilize transfer learning (e.g., when tested against a test dataset). Accordingly, in some embodiments, a non-transfer learning approach is utilized to generate a trained model.

In some embodiments, the trained model comprises 10 layers to 1,000,000 layers. In some embodiments, the model comprises 10 layers to 50 layers, 10 layers to 100 layers, 10 layers to 200 layers, 10 layers to 500 layers, 10 layers to 1,000 layers, 10 layers to 5,000 layers, 10 layers to 10,000 layers, 10 layers to 50,000 layers, 10 layers to 100,000 layers, 10 layers to 500,000 layers, 10 layers to 1,000,000 layers, 50 layers to 100 layers, 50 layers to 200 layers, 50 layers to 500 layers, 50 layers to 1,000 layers, 50 layers to 5,000 layers, 50 layers to 10,000 layers, 50 layers to 50,000 layers, 50 layers to 100,000 layers, 50 layers to 500,000 layers, 50 layers to 1,000,000 layers, 100 layers to 200 layers, 100 layers to 500 layers, 100 layers to 1,000 layers, 100 layers to 5,000 layers, 100 layers to 10,000 layers, 100 layers to 50,000 layers, 100 layers to 100,000 layers, 100 layers to 500,000 layers, 100 layers to 1,000,000 layers, 200 layers to 500 layers, 200 layers to 1,000 layers, 200 layers to 5,000 layers, 200 layers to 10,000 layers, 200 layers to 50,000 layers, 200 layers to 100,000 layers, 200 layers to 500,000 layers, 200 layers to 1,000,000 layers, 500 layers to 1,000 layers, 500 layers to 5,000 layers, 500 layers to 10,000 layers, 500 layers to 50,000 layers, 500 layers to 100,000 layers, 500 layers to 500,000 layers, 500 layers to 1,000,000 layers, 1,000 layers to 5,000 layers, 1,000 layers to 10,000 layers, 1,000 layers to 50,000 layers, 1,000 layers to 100,000 layers, 1,000 layers to 500,000 layers, 1,000 layers to 1,000,000 layers, 5,000 layers to 10,000 layers, 5,000 layers to 50,000 layers, 5,000 layers to 100,000 layers, 5,000 layers to 500,000 layers, 5,000 layers to 1,000,000 layers, 10,000 layers to 50,000 layers, 10,000 layers to 100,000 layers, 10,000 layers to 500,000 layers, 10,000 layers to 1,000,000 layers, 50,000 layers to 100,000 layers, 50,000 layers to 500,000 layers, 50,000 layers to 1,000,000 layers, 100,000 layers to 500,000 layers, 100,000 layers to 1,000,000 layers, or 500,000 layers to 1,000,000 layers. In some embodiments, the model comprises 10 layers, 50 layers, 100 layers, 200 layers, 500 layers, 1,000 layers, 5,000 layers, 10,000 layers, 50,000 layers, 100,000 layers, 500,000 layers, or 1,000,000 layers. In some embodiments, the model comprises at least 10 layers, 50 layers, 100 layers, 200 layers, 500 layers, 1,000 layers, 5,000 layers, 10,000 layers, 50,000 layers, 100,000 layers, or 500,000 layers. In some embodiments, the model comprises at most 50 layers, 100 layers, 200 layers, 500 layers, 1,000 layers, 5,000 layers, 10,000 layers, 50,000 layers, 100,000 layers, 500,000 layers, or 1,000,000 layers.

In some embodiments, a machine learning method comprises a trained model or classifier that is tested using data that was not used for training to evaluate its predictive ability. In some embodiments, the predictive ability of the trained model or classifier is evaluated using one or more performance metrics. These performance metrics include classification accuracy, specificity, sensitivity, positive predictive value, negative predictive value, measured area under the receiver operator curve (AUROC), mean squared error, false discover rate, and Pearson correlation between the predicted and actual values which are determined for a model by testing it against a set of independent cases. If the values are continuous, root mean squared error (MSE) or Pearson correlation coefficient between the predicted value and the measured values are two common metrics. For discrete classification tasks, classification accuracy, positive predictive value, precision/recall, and area under the ROC curve (AUC) are common performance metrics.

In some instances, an method has an AUROC of at least about 60%, 65%, 70%, 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an method has an accuracy of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an method has a specificity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an method has a sensitivity of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances, an method has a positive predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein. In some instances an method has a negative predictive value of at least about 75%, 80%, 85%, 90%, 95% or more, including increments therein, for at least about 50, 60, 70, 80, 90, 100, 110, 120, 130, 140, 150, 160, 170, 180, 190, or 200 independent cases, including increments therein.

### Computing Systems and Software

In some embodiments, a system as described herein is configured to provide a software application such as a polypeptide prediction engine. In some embodiments, the polypeptide prediction engine comprises one or more models for predicting at least one function or property based on input data such as a primary amino acid sequence. In some embodiments, a system as described herein comprises a computing device such as a digital processing device. In some embodiments, a system as described herein comprises a network element for communicating with a server. In some embodiments, a system as described herein comprises a server. In some embodiments, the system is configured to upload to and/or download data from the server. In some embodiments, the server is configured to store input data, output, and/or other information. In some embodiments, the server is configured to backup data from the system or apparatus.

In some embodiments, the system comprises one or more digital processing devices. In some embodiments, the system comprises a plurality of processing units configured to generate the trained model(s). In some embodiments, the system comprises a plurality of graphic processing units (GPUs), which are amenable to machine learning applications. For example, GPUs are generally characterized by an increased number of smaller logical cores composed of arithmetic logic units (ALUs), control units, and memory caches when compared to central processing units (CPUs). Accordingly, GPUs are configured to process a greater number of simpler and identical computations in parallel, which are amenable to the math matrix calculations common in machine learning approaches. In some embodiments, the system comprises one or more tensor processing units (TPUs), which are AI application-specific integrated circuits (ASIC) developed by Google for neural network machine learning. In some embodiments, the methods described herein are implemented on systems comprising a plurality of GPUs and/or TPUs. In some embodiments, the systems comprise at least 2, 3, 4, 5, 6, 7, 8, 9, 10, 15, 20, 30, 40, 50, 60, 70, 80, 90, or 100 or more GPUs or TPUs. In some embodiments, the GPUs or TPUs are configured to provide parallel processing.

In some embodiments, the system or apparatus is configured to encrypt data. In some embodiments, data on the server is encrypted. In some embodiments, the system or apparatus comprises a data storage unit or memory for storing data. In some embodiments, data encryption is carried out using Advanced Encryption Standard (AES). In some embodiments, data encryption is carried out using 128-bit, 192-bit, or 256-bit AES encryption. In some embodiments, data encryption comprises full-disk encryption of the data storage unit. In some embodiments, data encryption comprises virtual disk encryption. In some embodiments, data encryption comprises file encryption. In some embodiments, data that is transmitted or otherwise communicated between the system or apparatus and other devices or servers is encrypted during transit. In some embodiments, wireless communications between the system or apparatus and other devices or servers is encrypted. In some embodiments, data in transit is encrypted using a Secure Sockets Layer (SSL).

An apparatus as described herein comprises a digital processing device that includes one or more hardware central processing units (CPUs) or general purpose graphics processing units (GPGPUs) that carry out the device's functions. The digital processing device further comprises an operating system configured to perform executable instructions. The digital processing device is optionally connected to a computer network. The digital processing device is optionally connected to the Internet such that it accesses the World Wide Web. The digital processing device is optionally connected to a cloud computing infrastructure. Suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein.

Typically, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing.

A digital processing device as described herein either includes or is operatively coupled to a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, a system or method as described herein generates a database as containing or comprising input and/or output data. Some embodiments of the systems described herein are computer based systems. These embodiments include a CPU including a processor and memory which may be in the form of a non-transitory computer readable storage medium. These system embodiments further include software that is typically stored in memory (such as in the form of a non-transitory computer readable storage medium) where the software is configured to cause the processor to carry out a function. Software embodiments incorporated into the systems described herein contain one or more modules.

In various embodiments, an apparatus comprises a computing device or component such as a digital processing device. In some of the embodiments described herein, a digital processing device includes a display to display visual information. Non-limiting examples of displays suitable for use with the systems and methods described herein include a liquid crystal display (LCD), a thin film transistor liquid crystal display (TFT-LCD), an organic light emitting diode (OLED) display, an OLED display, an active-matrix OLED (AMOLED) display, or a plasma display.

A digital processing device, in some of the embodiments described herein includes an input device to receive information. Non-limiting examples of input devices suitable for use with the systems and methods described herein include a keyboard, a mouse, trackball, track pad, or stylus. In some embodiments, the input device is a touch screen or a multi-touch screen.

The systems and methods described herein typically include one or more non-transitory computer readable storage media encoded with a program including instructions executable by the operating system of an optionally networked digital processing device. In some embodiments of the systems and methods described herein, the non-transitory storage medium is a component of a digital processing device that is a component of a system or is utilized in a method. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

Typically the systems and methods described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages. The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

Typically, the systems and methods described herein include and/or utilize one or more databases. In view of the disclosure provided herein, those of skill in the art will recognize that many databases are suitable for storage and retrieval of baseline datasets, files, file systems, objects, systems of objects, as well as data structures and other types of information described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In other embodiments, a database is based on one or more local computer storage devices.

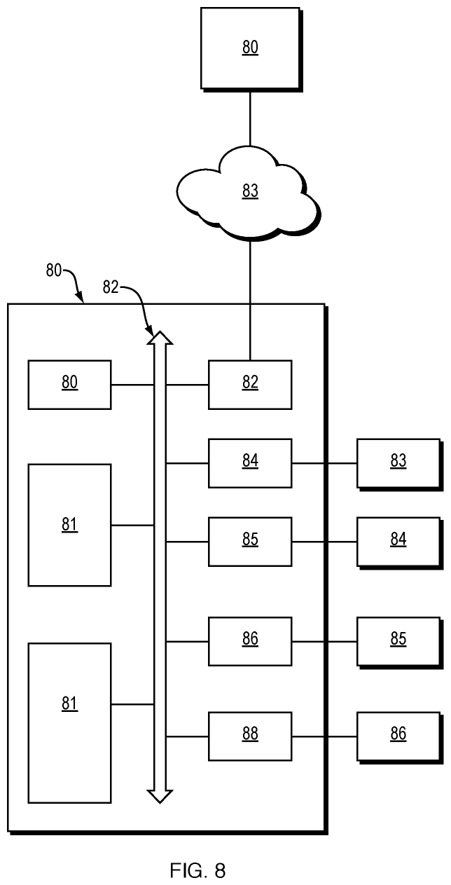

FIG. 8 shows an exemplary embodiment of a system as described herein comprising an apparatus such as a digital processing device 801. The digital processing device 801 includes a software application configured to analyze input data. The digital processing device 801 may include a central processing unit (CPU, also “processor” and “computer processor” herein) 805, which can be a single core or multi-core processor, or a plurality of processors for parallel processing. The digital processing device 801 also includes either memory or a memory location 810 (e.g., random-access memory, read-only memory, flash memory), electronic storage unit 815 (e.g., hard disk), communication interface 820 (e.g., network adapter, network interface) for communicating with one or more other systems, and peripheral devices, such as cache. The peripheral devices can include storage device(s) or storage medium 865 which communicate with the rest of the device via a storage interface 870. The memory 810, storage unit 815, interface 820 and peripheral devices are configured to communicate with the CPU 805 through a communication bus 825, such as a motherboard. The digital processing device 801 can be operatively coupled to a computer network (“network”) 830 with the aid of the communication interface 820. The network 830 can comprise the Internet. The network 830 can be a telecommunication and/or data network.

The digital processing device 801 includes input device(s) 845 to receive information, the input device(s) in communication with other elements of the device via an input interface 850. The digital processing device 801 can include output device(s) 855 that communicates to other elements of the device via an output interface 860.

The CPU 805 is configured to execute machine-readable instructions embodied in a software application or module. The instructions may be stored in a memory location, such as the memory 810. The memory 810 may include various components (e.g., machine readable media) including, but not limited to, a random access memory component (e.g., RAM) (e.g., a static RAM “SRAM”, a dynamic RAM “DRAM, etc.), or a read-only component (e.g., ROM). The memory 810 can also include a basic input/output system (BIOS), including basic routines that help to transfer information between elements within the digital processing device, such as during device start-up, may be stored in the memory 810.

The storage unit 815 can be configured to store files, such as primary amino acid sequences. The storage unit 815 can also be used to store operating system, application programs, and the like. Optionally, storage unit 815 may be removably interfaced with the digital processing device (e.g., via an external port connector (not shown)) and/or via a storage unit interface. Software may reside, completely or partially, within a computer-readable storage medium within or outside of the storage unit 815. In another example, software may reside, completely or partially, within processor(s) 805.

Information and data can be displayed to a user through a display 835. The display is connected to the bus 825 via an interface 840, and transport of data between the display other elements of the device 801 can be controlled via the interface 840.

Methods as described herein can be implemented by way of machine (e.g., computer processor) executable code stored on an electronic storage location of the digital processing device 801, such as, for example, on the memory 810 or electronic storage unit 815. The machine executable or machine readable code can be provided in the form of a software application or software module. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

In some embodiments, a remote device 802 is configured to communicate with the digital processing device 801, and may comprise any mobile computing device, non-limiting examples of which include a tablet computer, laptop computer, smartphone, or smartwatch. For example, in some embodiments, the remote device 802 is a smartphone of the user that is configured to receive information from the digital processing device 801 of the apparatus or system described herein in which the information can include a summary, input, output, or other data. In some embodiments, the remote device 802 is a server on the network configured to send and/or receive data from the apparatus or system described herein.

Some embodiments of the systems and methods described herein are configured to generate a database containing or comprising input and/or output data. A database, as described herein, is configured to function as, for example, a data repository for input and output data. In some embodiments, the database is stored on a server on the network. In some embodiments the database is stored locally on the apparatus (e.g., the monitor component of the apparatus). In some embodiments, the database is stored locally with data backup provided by a server.

### Certain Definitions

As used herein, the singular forms “a”, “an” and “the” include plural references unless the context clearly dictates otherwise. For example, the term “a sample” includes a plurality of samples, including mixtures thereof. Any reference to “or” herein is intended to encompass “and/or” unless otherwise stated.

The term “nucleic acid” as used herein generally refers to one or more nucleobases, nucleosides, or nucleotides. For example, a nucleic acid may include one or more nucleotides selected from adenosine (A), cytosine (C), guanine (G), thymine (T) and uracil (U), or variants thereof. A nucleotide generally includes a nucleoside and at least 1, 2, 3, 4, 5, 6, 7, 8, 9, 10, or more phosphate (PO3) groups. A nucleotide can include a nucleobase, a five-carbon sugar (either ribose or deoxyribose), and one or more phosphate groups. Ribonucleotides include nucleotides in which the sugar is ribose. Deoxyribonucleotides include nucleotides in which the sugar is deoxyribose. A nucleotide can be a nucleoside monophosphate, nucleoside diphosphate, nucleoside triphosphate or a nucleoside polyphosphate.

As used herein, the terms “polypeptide”, “protein” and “peptide” are used interchangeably and refer to a polymer of amino acid residues linked via peptide bonds and which may be composed of two or more polypeptide chains. The terms “polypeptide”, “protein” and “peptide” refer to a polymer of at least two amino acid monomers joined together through amide bonds. An amino acid may be the L-optical isomer or the D-optical isomer. More specifically, the terms “polypeptide”, “protein” and “peptide” refer to a molecule composed of two or more amino acids in a specific order; for example, the order as determined by the base sequence of nucleotides in the gene or RNA coding for the protein. Proteins are essential for the structure, function, and regulation of the body's cells, tissues, and organs, and each protein has unique functions. Examples are hormones, enzymes, antibodies, and any fragments thereof. In some cases, a protein can be a portion of the protein, for example, a domain, a subdomain, or a motif of the protein. In some cases, a protein can be a variant (or mutation) of the protein, wherein one or more amino acid residues are inserted into, deleted from, and/or substituted into the naturally occurring (or at least a known) amino acid sequence of the protein. A protein or a variant thereof can be naturally occurring or recombinant. A polypeptide can be a single linear polymer chain of amino acids bonded together by peptide bonds between the carboxyl and amino groups of adjacent amino acid residues. Polypeptides can be modified, for example, by the addition of carbohydrate, phosphorylation, etc. Proteins can comprise one or more polypeptides.

As used herein, the term “neural net” refers to an artificial neural network. An artificial neural network has the general structure of an interconnected group of nodes. The nodes are often organized into a plurality of layers in which each layer comprises one or more nodes. Signals can propagate through the neural network from one layer to the next. In some embodiments, the neural network comprises an embedder. The embedder can include one or more layers such as embedding layers. In some embodiments, the neural network comprises a predictor. The predictor can include one or more output layers that generate the output or result (e.g., a predicted function or property based on a primary amino acid sequence).

As used herein, the term “pretrained system” refers to at least one model trained on at least one data set. Examples of models can be linear models, transformers, or neural networks such as convolutional neural networks (CNNs). A pretrained system can include one or more of the models trained on one or more of the data sets. The system can also include weights, such as embedded weights for a model or neural network.

As used herein, the term “artificial intelligence” generally refers to machines or computers that can perform tasks in a manner that is “intelligent” or non-repetitive or rote or pre-programmed.

As used herein, the term “machine learning” refers to a type of learning in which the machine (e.g., computer program) can learn on its own without being programmed.

As used herein, the term “machine learning” refers to a type of learning in which the machine (e.g., computer program) can learn on its own without being programmed.

As used herein, the term “about” a number refers to that number plus or minus 10% of that number. The term “about” a range refers to that range minus 10% of its lowest value and plus 10% of its greatest value.

As used herein, the phrase “at least one of a, b, c, and d” refers to a, b, c, or d, and any and all combinations comprising two or more than two of a, b, c, and d.

### EXAMPLES

### Example 1: Building of a Model for all Protein Functions and Features

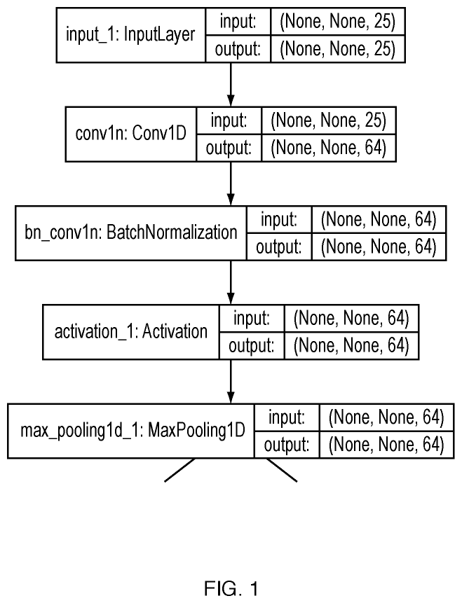

This example describes the building of the first model in transfer learning for specific protein functions or protein properties. The first model was trained on 58 million protein sequences from the Uniprot database (https://www.uniprot.org/), with 172,401+ annotations across 7 different functional representations (GO, Pfam, keywords, Kegg Ontology, Interpro, SUPFAM, and OrthoDB). The model was based on a deep neural network that follows the residual learning architecture. The input to the network was a protein sequence represented as a “one-hot” vector that encodes the sequence of amino acids as a matrix where each row contains exactly 1 non-zero entry which corresponds to the amino acid present at that residue. The matrix allowed for 25 possible amino acids to cover all canonical and non-canonical amino acid possibilities, and all proteins longer than 1000 amino acids were truncated to the first 1000 amino acids. The input was then processed by a 1-dimensional convolutional layer with 64 filters, followed by a batch normalization, a rectified linear (ReLU) activation function, and finally by a 1-dimensional max-pooling operation. This is referred to as the “input block” and is shown in FIG. 1.

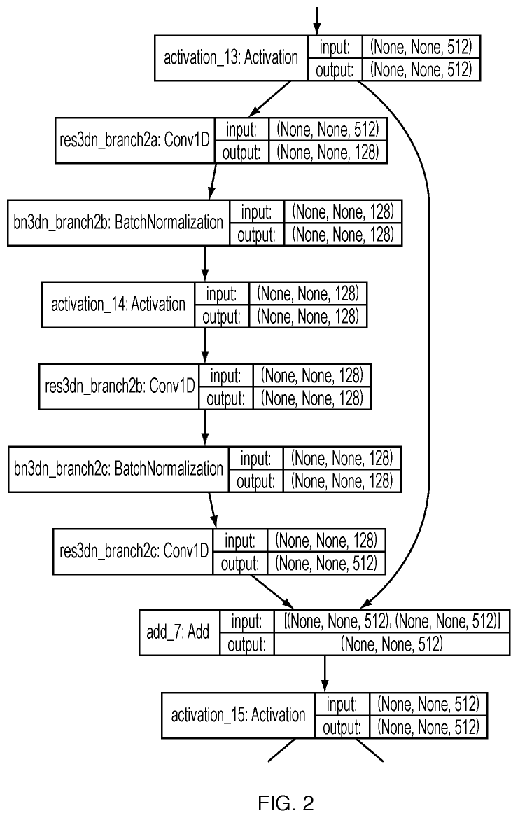

After the input block, a repeated series of operations known as an “identity block” and a “convolutional block” were performed. An identity block performed a series of 1-dimensional convolutions, batch normalizations, and ReLU activations to transform the input to the block, while preserving the shape of the input. The result of these transformations was then added back to the input and transformed using a ReLU activation, and was then passed on to subsequent layers/blocks. An example identity block is shown in FIG. 2.

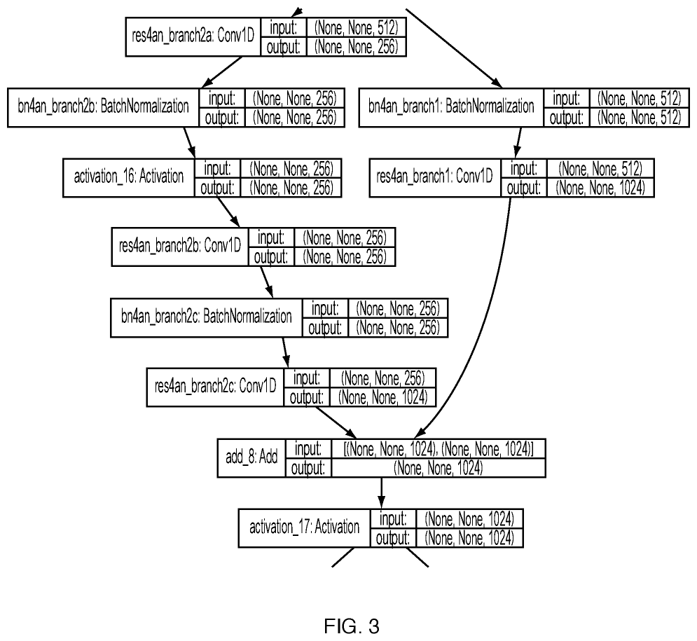

A convolutional block is similar to an identity block except that instead of the identity branch, it contains a branch with a single convolutional operation that resizes the input. These convolutional blocks are used to change the size (e.g., often to increase) of the network's internal representation of the protein sequence. An example of a convolutional block is shown in FIG. 3.

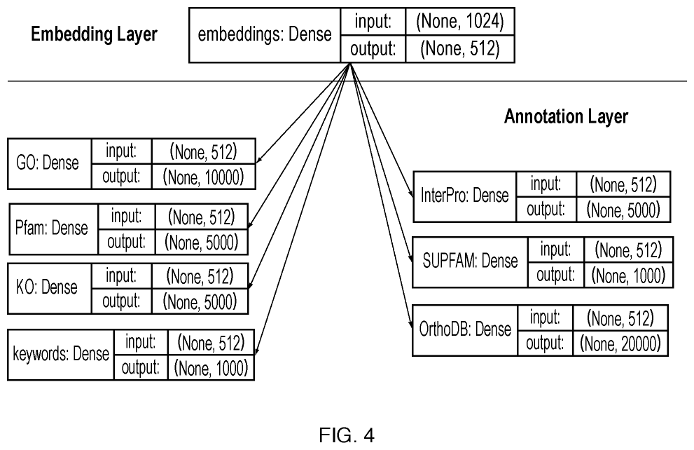

After the input block, a series of operations in the form of a convolutional block (to resize the representation) followed by 2-5 identity blocks were used to build the core of the network. This schema (convolutional block+multiple identity blocks) was repeated 5 times in total. Finally, a global average pooling layer followed by dense layer with 512 hidden units was performed to create the sequence embeddings. The embeddings can be viewed as a vector which lives in 512-dimensional space that encodes all of the information in the sequence that is relevant for function. Using the embeddings, the presence or absence of each of the 172,401 annotations was predicted using a linear model for each annotation. The output layer displaying this process is shown in FIG. 4.

The model was trained for 6 full passes over the 57,587,648 proteins in the training dataset using a variant of stochastic gradient descent known as Adam on a compute node with 8 V100 GPUs. Training took approximately one week. The trained model was validated using a validation data set composed of about 7 million proteins.

The network is trained to minimize the sum binary cross-entropy for each annotation, except for OrthoDB which used a categorical cross-entropy loss. Since some annotations are very rare, a loss-reweighting strategy improves performance. For each binary classification task, the loss from the minority class (e.g., the positive class) is up-weighted using the square-root of the inverse frequency of the minority class. This encourages the network to “pay attention” approximately equally to both positive and negative examples, even though most sequences are negative examples for the vast majority of annotations.

The final model results in an overall weighted F1 accuracy of 0.84 (Table 1) to predict any label across the 7 different tasks from primary protein sequence alone. F1 is a measure of accuracy that the harmonic mean of precision and recall and is perfect when at 1 and total failure at 0. The macro and micro average accuracies are shown in Table 1. For a macro-average, the accuracy is calculated independently for each class and then the average is determined. This approach treats all classes equally. The micro-average accuracy aggregates the contributions of all classes to calculate the average metric.

TABLE 1
Predictive accuracy of the first model

| Source | Macro | Micro |
|---|---|---|
| GO | 0.42 | 0.75 |
| InterPro | 0.63 | 0.83 |
| keywords | 0.80 | 0.88 |
| KO | 0.23 | 0.25 |
| OrthoDB | 0.76 | 0.91 |
| Pfam | 0.63 | 0.82 |
| SUPFAM | 0.77 | 0.91 |

### Example 2: Deep Neural Network Analysis Technique for Protein Stability

This example describes the training of the second model to predict a specific protein property of protein stability directly from a primary amino acid sequence. The first model described in Example 1 is used as a starting point for the training of the second model.

The data input for the second model is obtained from Rocklin et al., Science, 2017 and includes 30,000 mini proteins that had been evaluated in a high-throughput yeast display assay for protein stability. Briefly, to generate the data input for the second model in this example, proteins were assayed for stability by using a yeast display system with each assayed protein genetically fused to an expression tag that can be fluorescently labeled. Cells were incubated with varying concentrations of protease. Those cells displaying stable proteins were isolated by fluorescence-activated cell sorting (FACS), and the identity of each protein was determined by deep sequencing. A final stability score was determined that indicates the difference between the measured EC50 and the predicted EC50 of that sequence in the unfolded state.

This final stability score is used as the data input for the second model. The real-valued stability scores for 56,126 amino acid sequences were extracted from the published supplementary data of Rocklin et al., then shuffled and randomly assigned to either a training set of 40,000 sequences or an independent test set of 16,126 sequences.

The architecture from the pretrained model of Example 1 is adjusted by removing the output layers of annotation prediction and adding a densely connected, 1-dimensional output layer with linear activation function, in order to fit to the per-sample protein stability value. Using a batch size of 128 sequences and Adam optimization with a learning rate of 1×10⁻⁴, the model is fit to 90% of the training data and validated with the remaining 10%, minimizing mean squared error (MSE) for up to 25 epochs (stopping early if validation loss increased for two consecutive epochs). This procedure is repeated both for a pretrained model, which is a transfer learning model with pretrained weights, as well as for an identical model architecture with randomly initialized parameters (the “naïve” model). For baseline comparison, a linear regression model with L2 regularization (the “ridge” model) is fit to the same data. Performance is evaluated via both MSE and Pearson correlation for predicted versus actual values in the independent test set. Next, a “learning curve” is created by drawing 10 random samples from the training set at sample sizes of 10, 50, 100, 500, 1000, 5000, and 10000, and repeats the above train/test procedure for each model.

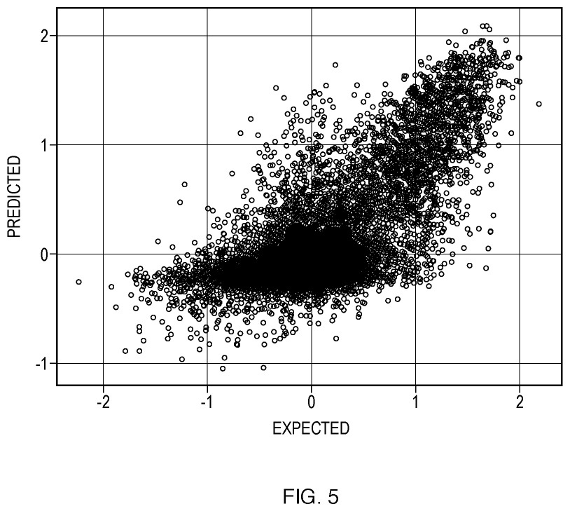

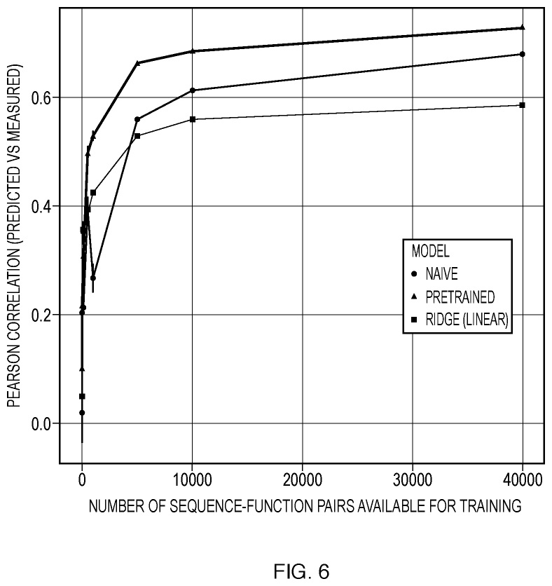

After training the first model as described in Example 1 and using it as a starting point for the training of the second model as described in the current Example 2, a Pearson correlation of 0.72 and MSE of 0.15 between the predicted and expected stability is demonstrated (FIG. 5) with the predictive capability up 24% from standard linear regression model. The learning curve of FIG. 6 demonstrates the high relative accuracy of the pretrained model at low sample sizes, which is sustained as the training set grows. Compared with the naïve model, the pretrained model requires fewer samples to achieve an equivalent level of performance, though the models appear to converge at high samples sizes as expected. Both deep learning models outperformed the linear model at a certain sample size, as the performance in the linear model eventually saturates.

### Example 3: Deep Neural Network Analysis Technique for Protein Fluorescence

This example describes the training of the second model to predict the specific protein function, of fluorescence directly from primary sequence.

The first model described in Example 1 is used as a starting point for the training of the second model. In this example, the data input for the second model is from Sarkisyan et al., Nature, 2016 and included 51,715 labeled GFP variants. Briefly, GFP activity was assayed using fluorescence-activated cell sorting to sort the bacteria expressing each variant into eight populations with different brightness of 510 nm emission.

The architecture from the pretrained model of Example 1 is adjusted by removing the output layers of annotation prediction and adding a densely connected, 1-dimensional output layer with sigmoid activation function, in order to classify each sequence as either fluorescing or not fluorescing. Using a batch size of 128 sequences and Adam optimization with a learning rate of 1×10⁻⁴, the model is trained to minimize binary cross entropy for 200 epochs. This procedure is repeated both for the transfer learning model with pretrained weights (the “pretrained” model), as well as for an identical model architecture with randomly initialized parameters (the “naïve” model). For baseline comparison, a linear regression model with L2 regularization (the “ridge” model) is fit to the same data.

The full data is split into a training and validation set, where the validation data were the top 20% brightest proteins, and the training set is the bottom 80%. To estimate how the transfer learning model might improve upon non-transfer learning approaches, the training dataset is sub-sampled to create sample sizes of 40, 50, 100, 500, 1000, 5000, 10000, 25000, 40000, and 48000 sequences. Random sampling is carried out for 10 realizations of each sample size from the full training dataset to measure performance and variability of each method. The primary metric of interest is positive predictive value, which is the percentage of true positives among all positive predictions from the model.

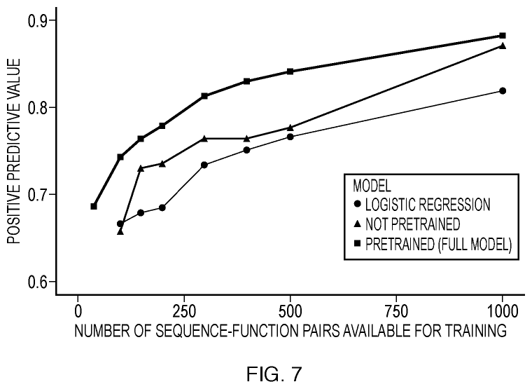

The addition of the transfer learning both increased overall positive predictive value, but also allowed prediction capabilities with less data than any other method (FIG. 7). For example, with 100 sequence-function GFP pairs as the input data to the second model, the addition of the first model for training resulted in 33% reduction in incorrect predictions. In addition, with only 40 sequence-function GFP pairs as the input data to the second model, the addition of the first model for training resulted in 70% positive predictive value, while the second model alone or a standard logistic regression model were undefined with 0 positive predictive value.

### Example 4: Deep Neural Network Analysis Technique for Protein Enzymatic Activity

This example describes the training of the second model to predict protein enzymatic activity directly from a primary amino acid sequence. The data input for the second model is from Halabi et al., Cell, 2009 and included 1,300 S1A serine proteases. Data description, quoted from the paper is as follows: “Sequences comprising the S1A, PAS, SH2, and SH3 families were collected from the NCBI nonredundant database (release 2.2.14, May-07-2006) through iterative PSI-BLAST (Altschul et al., 1997) and aligned with Cn3D (Wang et al., 2000) and ClustalX (Thompson et al., 1997) followed by standard manual adjustment methods (Doolittle, 1996).” Using this data, the second model was trained with the goal of predicting primary catalytic specificity from the primary amino acid sequence for the following categories: trypsin, chymotrypsin, granzyme, and kallikrein. There are a total 422 sequences for these 4 categories. Importantly, none of the models used a multiple sequence alignment, which demonstrated that this task was possible without requiring a multiple sequence alignment.

The architecture from the pretrained model of Example 1 is adjusted by removing the output layers of annotation prediction and adding a densely connected, 4-dimensional output layer with softmax activation function, in order to classify each sequence into 1 of the 4 possible categories. Using a batch size of 128 sequences and Adam optimization with a learning rate of 1×10⁻⁴, the model is fit to 90% of the training data and validated with the remaining 10%, minimizing categorical cross-entropy for up to 500 epochs (stopping early if validation loss increased for ten consecutive epochs). This entire process is repeated 10 times (known as 10-fold cross validation) to assess accuracy and variability for each model. This is repeated both for the pretrained model, which is the transfer learning model with pretrained weights, as well as for an identical model architecture with randomly initialized parameters (the “naïve” model). For baseline comparison, a linear regression model with L2 regularization (the “ridge” model) is fit to the same data. Performance is evaluated classification accuracy on the withheld data in each fold.

After training the first model as described in Example 1 and using it as a starting point for the training of the second model as described in the current Example 2, the results demonstrated a median classification accuracy of 93% using the pretrained model compared to 81% with the naïve model and 80% using linear regression. This is shown in Table 2.

TABLE 2
Classification accuracy on S1A serine protease data

| Model | Median Accuracy |
|---|---|
| Logistic Regression | 80% |
| Naïve Model | 81% |
| Pretrained Model | 92% |

### Example 5: Deep Neural Network Analysis Technique for Protein Solubility

Many amino acid sequences result in structures that aggregate in solution. Reducing the tendency of the amino acid sequences to aggregate (e.g., improving solubility), is a goal to design better therapeutics. Therefore, models for predicting aggregation and solubility directly from sequence are important tools toward this end. This example describes the self-supervised pretraining of a transformer architecture and subsequent fine-tuning of the model to predict amyloid-beta (Aβ) solubility via a readout of the inverse property, protein aggregation. The data is measured using an aggregation assay for all possible single point mutations in a high-throughput deep mutational scan. Gray et al., “Elucidating the Molecular Determinants of AB Aggregation with Deep Mutational Scanning” in G3, 2019, includes data used to train the present model, in at least one example. However, in some embodiments, other data can be used for training. In this example, the effectiveness of transfer learning is demonstrated using a different encoder architecture from previous examples, in this case using a transformer instead of a convolutional neural network. Transfer learning improves generalization of the model to protein positions unseen in the training data.

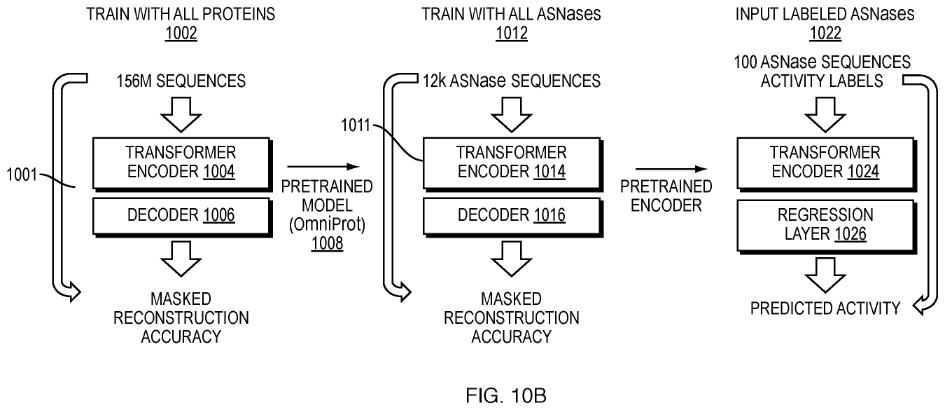

In this example, data is gathered and formatted as a set of 791 sequence-label pairs. The labels are the mean of real-valued aggregation assay measurements over multiple replicates for each sequence. The data is split into train/test sets in a 4:1 ratio by two methods: (1) randomly, with each labeled sequence assigned to either the training, validation, or test set, or (2) by residue, with all sequences with mutations at a given position grouped together in either the training or the test set, such that the model is isolated from (e.g., never be exposed to) data from certain randomly selected positions during training, but is forced to predict outputs at these unseen positions on the held-out test data. FIG. 11 illustrates an example embodiment of splitting by protein position.

This example employs the transformer architecture of the BERT language model for predicting properties of proteins. The model is trained in a “self-supervised” manner, such that certain residues of the input sequence are masked, or hidden, from the model, and the model is tasked with determining the identity of the masked residues given the unmasked residues. In this example, the model is trained with the full set of over 156 million protein amino acid sequences available for download from the UniProtKB database at the time of model development. For each sequence, 15% of the amino acid positions are randomly masked from the model, the masked sequence is converted into the “one-hot” input format described in Example 1, and the model is trained to maximize the accuracy of masked prediction. A person having ordinary skill in the art can understand that Rives et al., “Biological Structure and Function Emerge from Scaling Unsupervised Learning to 250 Million Protein Sequences,” http://dx.doi.org/10.1101/622803, 2019 (hereinafter “Rives”) describes other applications.

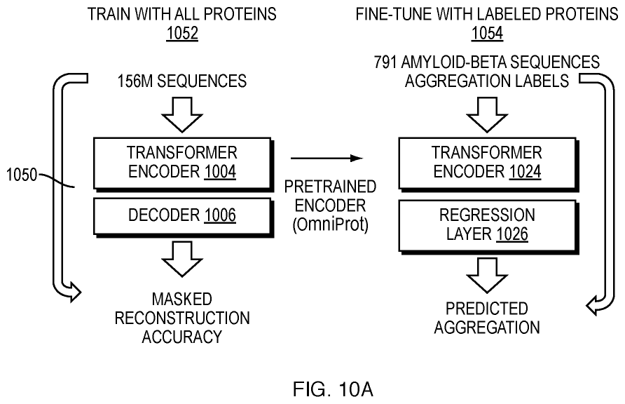

FIG. 10A is block diagram 1050 illustrating an example embodiment of the present disclosure. The diagram 1050 illustrates training Omniprot, one system that can implement the methods described in the present disclosure. Omniprot can refer to a pretrained transformer. It can be appreciated that training of Omniprot can be similar in aspects to Rives et al., but has variations as well. First, sequences and corresponding annotations having properties of the sequences (predicted functions or other properties) pretrain 1052 a neural network/model of Omniprot. These sequences are a large set of data, and in this example are 156 million sequences. Then, a smaller set of data, the specific library measurements, fine-tune 1054 Omniprot. In this particular example, the smaller set of data is 791 amyloid-beta sequences aggregation labels. However, a person having ordinary skill in the art can recognize that other numbers of sequences and labels, as well as other types, can be employed. Once finetuned, the Omniprot database can output a predicted function of sequences.

At a more detailed level, the transfer learning method fine-tunes the pretrained model for a protein aggregation prediction task. The decoder from the transformer architecture is removed, which reveals an L×D dimension tensor as an output from the remaining encoder, where L is the length of the protein and the embedding dimension D is a hyperparameter. This tensor is reduced to a D-dimensional embedding vector by calculating the mean over the length dimension L. Then, a new densely connected, 1-dimensional output layer with linear activation function is added, and weights for all layers in the model are fit to the scalar aggregation assay values. For baseline comparison, both a linear regression model with L2 regularization and a naïve transformer (using randomly initialized rather than pretrained weights) are also fit to the training data. Performance for all models are evaluated using Pearson correlation of predictions vs true labels for the held-out test data.

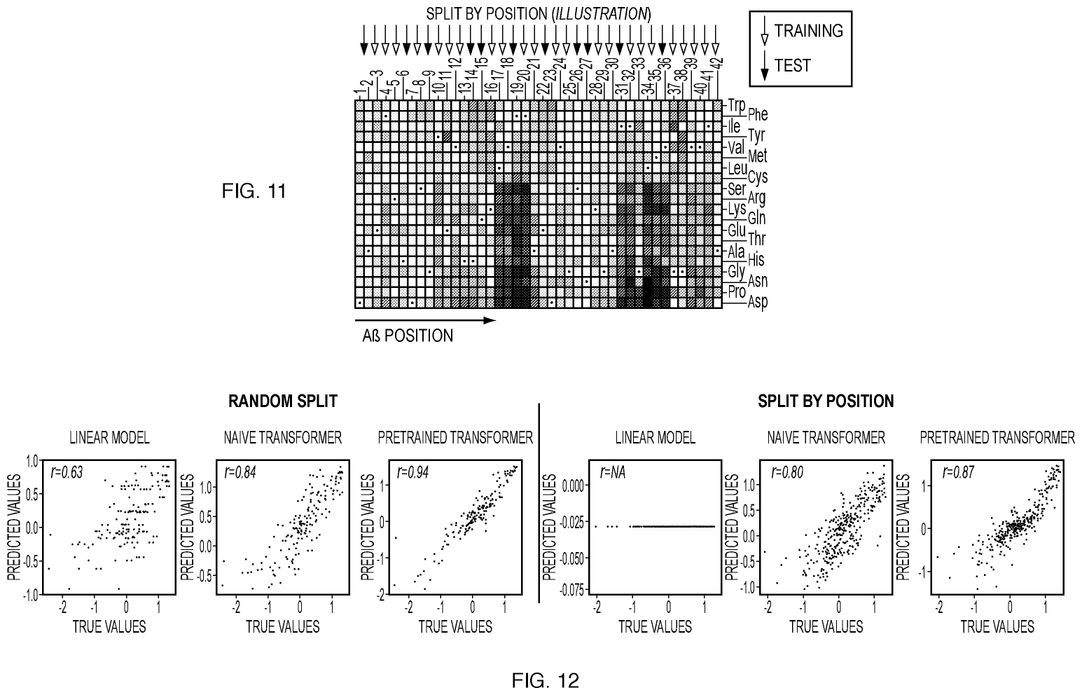

FIG. 12 illustrates example results of linear, naïve, and pretrained transformer results using a random split and a split by position. For all three models, splitting the data by position is a more difficult task, with performance dropping using all types of models. A linear model is unable to learn from the data in the position-based split, due to the nature of the data. The one-hot input vector has no overlaps between the train and test set for any particular amino acid variant. Both transformer models (e.g., Naïve transformer and Pretrained transformer), however, are able to generalize rules of protein aggregation from one set of positions to another set of positions unseen in the training data, with only a small loss in accuracy as compared to a random split of the data. The Naïve transformer has an r=0.80, and the pretrained transformer has an r=0.87. Furthermore, for both types of data splits, the pretrained transformer had considerably higher accuracy than the naïve model, demonstrating the power of transfer learning for proteins with a completely different deep learning architecture to the previous examples.

### Example 6: Successive Targeted Pretraining for Enzyme Activity Prediction

L-Asparaginase is a metabolic enzyme that converts the amino acid asparagine to aspartate and ammonium. While humans naturally produce this enzyme, a high-activity bacterial variant (derived from Escherichia coli or Erwinia chrysanthemi) is used to treat certain leukemias by direct injection into the body. Asparaginase works by removing L-asparagine from the bloodstream, killing the cancer cells which depend on the amino acid.

A set of 197 naturally occurring sequence variants of Type II asparaginase are assayed with the goal of developing a predictive model of enzyme activity. All sequences are ordered as cloned plasmids, expressed in E coli, isolated, and assayed for maximum enzymatic rate of the enzyme as follows: 96-well high binding plates are coated with anti-6His tag antibody. The wells are then washed and blocked using BSA blocking buffer. After blocking, the wells are washed again and then incubated with appropriately diluted E. coli lysate containing the expressed His-tagged ASNase. After 1 hour, the plates are washed and the asparaginase activity assay mixture (from Biovision kit K754) is added. Enzyme activity is measured by spectrophotometry at 540 nm, with reads taken every minute for 25 minutes. To determine the rate of each sample, the highest slope over a 4 minute window is taken as the maximum instantaneous rate for each enzyme. Said enzymatic rate is an example of a protein function. These activity-labeled sequences were separated into a 100-sequence training set and a 97-sequence test set.

FIG. 10B is a block diagram 1000 illustrating an example embodiment of the method of the present disclosure. In theory, a subsequent round of unsupervised fine-tuning of the pretrained model from Example 5, using all known asparaginase-like proteins, improves the predictive performance of the model in a transfer learning task on a small number of measured sequences. The pretrained transformer model of Example 5, having been initially trained on the universe of all known protein sequences from UniProtKB, is further fine-tuned on the 12,583 sequences annotated with the InterPro family IPR004550, “L-asparaginase, type II”. This is a two-step pretraining process, wherein both steps apply the same self-supervised method of Example 5.

A first system 1001, having a transformer encoder and decoder 1006, is trained using a set of all proteins. In this example, 156 million protein sequences are employed, however, a person having ordinary skill in the art can appreciate that other amounts of sequences can be used. A person having ordinary skill in the art can further appreciate that the size of the data used to train model 1001 is larger than the size of the data used to train the second system 1011. The first model generates a pretrained model 1008, which is sent to the second system 1011.

The second system 1011 accepts the pretrained model 1008, and trains the model with the smaller data set of ASNase sequences 1012. However, a person having ordinary skill in the art can recognize that other data sets can be used for this fine-tuning training. The second system 1011 then applies the transfer learning method to predict activity by replacing the decoder layer 1016 with a linear regression layer 1026, and further training the resulting model to predict scalar enzymatic activity values 1022 as a supervised task. The labeled sequences are split randomly into training and test sets. The model is trained on the training set of 100 activity-labeled asparaginase sequences 1022, and the performance is then evaluated on a held-out test set. As theorized, transfer learning with a second pretraining step—utilizing all available sequences in the protein family—produced a notable increase in predictive accuracy in the low data setting, that is, when the second trainings had less or considerably less data than the initial training.

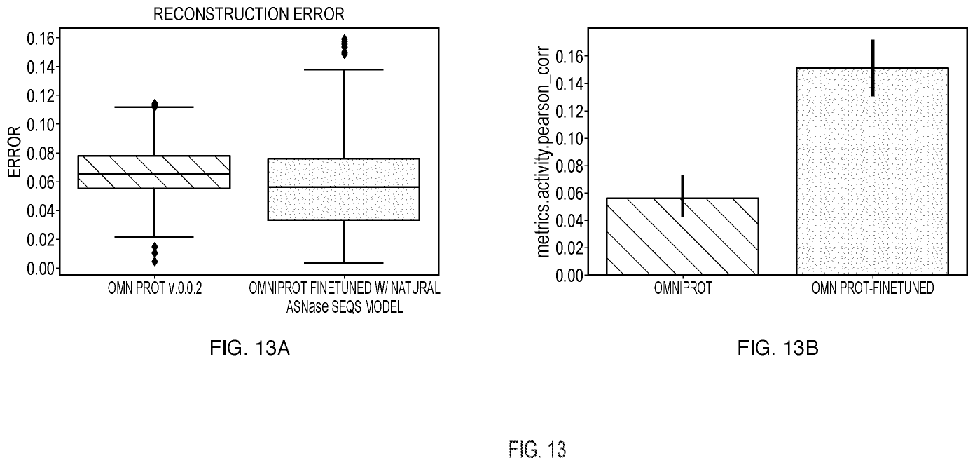

FIG. 13A is a graph illustrating reconstruction error for masked prediction of 1000 unlabeled asparaginase sequences. FIG. 13A illustrates that the reconstruction error after the second round of pretraining for asparaginase proteins (left) is reduced compared to the Omniprot finetuned with natural asnase sequence model (right). FIG. 13B is a graph illustrating predictive accuracy on the 97 held-out activity-labeled sequences, after training with only 100 labeled sequences. The Pearson correlation of measured activity vs model predictions is notably improved with the two-step pretraining, over the single (OmniProt) pretraining step.

In the above description and examples, a person having ordinary skill in the art can recognize that while particular numbers of sample sizes, iterations, epochs, batch sizes, learning rates, accuracies, data input sizes, filters, amino acids sequences, and other figures may be adjusted or optimized. While particular embodiments are described in the examples, the numbers listed in the examples are non-limiting.

While preferred embodiments of the present invention have been shown and described herein, it will be understood to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. While example embodiments have been particularly shown and described, it will be understood by those skilled in the art that various changes in form and details may be made therein without departing from the scope of the embodiments encompassed by the appended claims.

The teachings of all patents, published applications and references cited herein are incorporated by reference in their entirety.

1. A method of modeling a desired protein property comprising:
(a) providing a first pretrained system comprising a first neural net embedder and a first neural net predictor, the first neural net predictor of the pretrained system being different from the desired protein property;
(b) transferring at least a part of the first neural net embedder of the pretrained system to a second system, the second system comprising a second neural net embedder and a second neural net predictor, the second neural net predictor of the second system providing the desired protein property; and
(c) analyzing, by the second system, a primary amino acid sequence of a protein analyte in order to generate a prediction of the desired protein property for the protein analyte.

2-6. (canceled)

7. The method of claim 1, wherein the amino acid sequences include annotations across one or more functional representations including at least one of GP, Pfam, keyword, Kegg Ontology, Interpro, SUPFAM, or OrthoDB.

8. (canceled)

9. The method of claim 1, wherein the second model has an improved performance metric relative to a model trained without using the transferred embedder of the first model.

10. The method of claim 1, wherein the first or second systems are optimized by Adam, RMS prop, stochastic gradient descent (SGD) with momentum, SGD with momentum and Nestrov accelerated gradient, SGD without momentum, Adagrad, Adadelta, or NAdam.

11. The method of claim 1, wherein the first and the second model can be optimized using any of the follow activation functions: softmax, elu, SeLU, softplus, softsign, ReLU, tanh, sigmoid, hard_sigmoid, exponential, PReLU, and LeaskyReLU, or linear.

12. (canceled)

13. The method of claim 1, wherein at least one of the first or second system utilizes a regularization selected from: early stopping, L1-L2 regularization, skip connections, or a combination thereof, wherein the regularization is performed on 1, 2, 3, 4, 5, or more layers.

14-15. (canceled)

16. The method of claim 1, wherein a second model of the second system comprises a first model of the first system in which the last layer of the first model is removed.

17. The method of claim 16, wherein 2, 3, 4, 5, or more layers of the first model are removed in a transfer to the second model.

18. The method of claim 17, wherein the transferred layers are frozen during the training of the second model.

19. The method of claim 17, wherein the transferred layers are unfrozen during the training of the second model.

20. (canceled)

21. The method of claim 1, wherein the neural net predictor of the second system predicts one or more of protein binding activity, nucleic acid binding activity, protein solubility, and protein stability.

22. The method of claim 1, wherein the neural net predictor of the second system predicts at least one of protein fluorescence and enzymatic activity.

23. (canceled)

24. A computer implemented method for identifying a previously unknown association between an amino acid sequence and a protein function comprising:
(a) generating, with a first machine learning software module, a first model of a plurality of associations between a plurality of protein properties and a plurality of amino acid sequences;
(b) transferring the first model or a portion thereof to a second machine learning software module;
(c) generating, by the second machine learning software module, a second model comprising at least a portion of the first model; and
(d) identifying, based on the second model, the previously unknown association between the amino acid sequence and the protein function.

25. The method of claim 24, wherein the amino acid sequence comprises a primary protein structure.

26. The method of claim 24, wherein the amino acid sequence causes a protein configuration that results in the protein function.

27. The method of claim 24, wherein the protein function comprises at least one of fluorescence, enzymatic activity, nuclease activity, and protein stability.

28-32. (canceled)

33. The method of claim 24, wherein the plurality of amino acid sequences forms a primary protein structure, a secondary protein structure, and a tertiary protein structure for a plurality of proteins.

34. The method of claim 24, wherein the first model is trained on input data comprising one or more of a multidimensional tensor, a representation of 3-dimensional atomic positions, an adjacency matrix of pairwise interactions, and a character embedding.

35. The method of claim 24, further comprising inputting to the second machine learning module, at least one of data related to a mutation of a primary amino acid sequence, a contact map of an amino acid interaction, a tertiary protein structure, and a predicted isoform from alternatively spliced transcripts.

36-63. (canceled)

64. A method modeling a desired protein property comprising:
training a first system with a first set of data, the first system comprising a first neural net transformer encoder and a first decoder, the first decoder of the pretrained system being configured to generate an output different from the desired protein property;
transferring at least a part of the first transformer encoder of the pretrained system to a second system, the second system comprising a second transformer encoder and a second decoder;
training the second system with a second set of data, the second set of data comprising a set of proteins representing a smaller number of classes of proteins than the first set, wherein the classes of proteins include one or more of: (a) classes of proteins within the first set of data, and (b) classes of proteins excluded from the first set of data; and
analyzing, by the second system, a primary amino acid sequence of a protein analyte, thereby generating a prediction of the desired protein property for the protein analyte.

65-69. (canceled)
